(19) United States
(12) Patent Application Publication
(10) Pub. No.: US 2020/0030368 A1
(43) Pub. Date: Jan. 30, 2020

(54) A POLYMER NETWORK, METHOD FOR PRODUCTION, AND USES THEREOF

(71) Applicant: JAWAHARLAL NEHRU CENTRE FOR ADVANCED SCIENTIFIC RESEARCH, Jakkur, Bangalore (IN)

(72) Inventors: Jayanta HALDAR, Bangalore (IN); Jiaul HOQUE, Bangalore (IN)

(21) Appl. No.: 16/321,674

(22) PCT Filed: Jul. 28, 2017
(86) PCT No.: PCT/IN2017/050311

§ 371 Date: Jan. 29, 2019

(30) Foreign Application Priority Data
Jul. 29, 2016 (IN) 201641026116

(51) Int. Cl. A61K 31/738; C08B 37/08; C08B 37/02; A61K 9/06; A61K 33/38; A61P 31/04

(52) U.S. Cl. CPC A61K 31/738; C08B 37/003; A61P 31/04; A61K 9/06; A61K 33/38; C08B 37/0021

(57) ABSTRACT

The present disclosure relates to a polymer network comprising a compound of Formula I cross-linked to a compound selected from the group consisting of a compound of Formula II; hyaluronate aldehyde, alginate aldehyde, dextran aldehyde, starch aldehyde, and chitosan aldehyde. It also relates to a process of preparing the polymer network. The present disclosure further relates to compositions comprising the polymer network and methods of preventing conditions and diseases that are caused by micro-organism. The present disclosure still further relates to a biocompatible antimicrobial hydrogel, a process for preparing the hydrogel, and methods of using the same, including a variety of tissue-related applications in which rapid adhesion to the tissue and gel formation is desired, as well as local delivery of pharmaceutical drugs to a site of application.

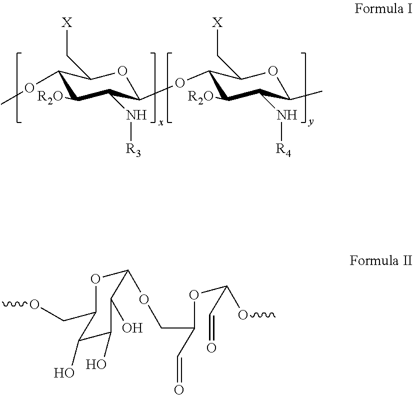

### TECHNICAL FIELD

The present disclosure relates to a polymer network and a process of preparing the polymer network. The present disclosure also relates to compositions and methods of preventing conditions and diseases that are caused by microorganism.

The present disclosure further relates to a biocompatible antimicrobial hydrogel, a process for preparing the hydrogel, and methods of using the same, including a variety of tissue-related applications in which rapid adhesion to the tissue and gel formation is desired, as well as local delivery of pharmaceutical drugs to a site of application.

### BACKGROUND

Infections at the surgical site result in prolonged wound healing, abscess formation and in severe cases whole body inflammation also known as sepsis. These infections are a significant clinical and financial burden on patients specially who are readmitted, often into intensive care units (ICUs), and are at higher risk of further complications.¹ Incision sites and dead spaces at the surgical sites are fertile infection locales, especially those in non-vascularised areas where the immune system has difficulty in detecting the infection, as well as those in areas of high adipose content that are nutrient rich for bacteria.² For patients undergoing medical procedures, surgical site infections are the most common type of infection encountered in the nosocomial environment.^{3,4} Bioadhesive materials are used as wound sealants and void fillers in clinical settings and generally adhere to tissue by forming chemical cross-links, or by mechanically fixing themselves to components of the extracellular matrix (ECM) in-situ.⁵⁻¹⁰ Such in situ gel-forming compositions are convenient to use since they can be administered as liquids from a variety of different devices, and are adaptable for administration to any site, since they are not preformed.^{11,12} However with fibrin-based adhesives, infection still remains a major concern since these sealants are not inherently antibacterial. Materials which can be applied to the damaged tissue during surgery that act as adhesive as well as thwart infection would thus be clinically useful. Moreover, the adhesive materials are also required to carry out other functions at the wound sites such as healing wounds, stopping unwanted bleeding, etc. Thus, an antibacterial bioadhesive that also acts as hemostatic and preferably wound healing material would be ideal for clinical use.

The most common tissue adhesives used currently in the market are fibrin sealant based products. In this system the components of the natural clotting factors, fibrinogen and thrombin, react mimicking the final stage of the body's natural clotting mechanism. The resulting fibrin clot or film adheres to the tissues to stop bleeding and improve the wound healing. The bond strengths of these products are not sufficient to hold tissues in approximation without the use of mechanical closures such as staples or sutures. Poor adhesive strength makes these hydrogels as poor bioadhesives. More importantly, these bioadhesive injectable hydrogel as sealant or void filler are not inherently antimicrobial or poorly antimicrobial. Cyanoacrylate products have been used to close skin breaks. When applied to tissue, the cyanoacrylate monomer undergoes an exothermic hydroxylation reaction that results in polymerization of the adhesive. However, inflammation, tissue necrosis, granule formation, and wound breakdown can occur when cyanoacrylates are implanted subcutaneously. The process is toxic due to the by-products of degradation, cyanoacetate and formaldehyde. The cured polymer is brittle and presents a barrier to tissue regrowth. More importantly, these bioadhesives are poorly antibacterial. Polyethylene glycol (PEG) products are on the market but their strength is fairly low, even with photopolymerization, and most products require mixing prior to use. Surgeon acceptance has apparently been slow even with the relative biological safety of the products. Also, these bioadhesives are not inherently antibacterial.

In an invention, a hydrogel with immobilized and encapsulated cells formed by cross-linking neutral chitosan with a bifunctional aldehyde containing polymer or aldehyde-treated hydroxyl-containing polymer has been reported to aid tissue regeneration or wound-healing at the surgical site.¹³ In another invention, a hydrogel comprises cross-linked derivatives of chitosan and dextran polymers was reported for use in wound healing, particularly for reducing post-surgical adhesions.¹⁴ Despite these efforts, surgical site infection still remains a major concern in surgery because of the lack of innate antibacterial activity of these hydrogel materials.

In a literature report, a chitosan dextran-based (CD) hydrogel developed for use in endoscopic sinus surgery was tested for antimicrobial activity in-vitro against a range of pathogenic microorganisms.¹⁵ However, the hydrogel is poorly antibacterial.

In another report, a polyethylenimine (PEI)-dextran based antibacterial injectable hydrogel was developed where polydextran aldehyde was used as bioadhesive and PEI was used as antibacterial component.¹⁶ However, polyethylenimine is not biocompatible and biodegradable and thus poses a significant threat to human life if used as an antibacterial material.^{17,18}

WO 2004006961 describes a gel for immobilizing and encapsulating cells formed by cross-linking neutral chitosan with a bifunctional multifunctional aldehyde or aldehyde-treated hydroxyl-containing polymer.

WO 2009028965 discloses a chitosan dextran-based (CD) hydrogel for use in endoscopic sinus surgery.

Giano et. al describes polyethylenimine (PEI)-dextran based injectable hydrogel where PEI was used as antibacterial component and polydextran aldehyde was used as bioadhesive.¹⁶

Further, U.S. Pat. Nos. 4,921,949 and 4,822,598 disclose that the HTCC, derived from chitosan, can be used as preservatives in cosmetic formulations. U.S. Pat. No. 6,306,835 describes the use of HTCC as antibacterial agent.

Despite these efforts, cytotoxicity of the hydrogel materials and the surgical site infections still remain major concerns.

Further at present, higher doses of antibiotics are administered to prevent or cure the infection at the surgical site. However, unwanted toxicity as well as development of resistance in bacteria has impacted the extensive uses of antibiotics¹⁹⁻²¹.

Therefore, there is a great need for new materials with efficacy for prevention of infections that can be used to improve surgical outcomes. In addition, the non-toxic antimicrobial materials with improved bioadhesive, wound healing and hemostatic abilities would be ideal for clinical applications.

### REFERENCES

1. Owens, C. D.; Stoessel, K. J. Hosp. Infect. 2008, 70, 3.
2. Soper, D. E.; Bump, R. C.; Hurt, W. G. Am. J. Obstet. Gynecol. 1995, 173, 465.
3. Mangram, A. J.; Horan, T. C.; Pearson, M. L.; Silver, L. C.; Jarvis, W. R. Infect. Control Hosp. Epidemiol. 1999, 20, 250.
4. Anderson, D. J.; Podgorny, K.; Berrios-Torres, S. I.; Bratzler, D. W.; DO, Dellinger, E. P.; Greene, L.; Nyquist, A.; Saiman, L.; Yokoe, D. S.; Maragakis, L. L.; Kaye, K. S. Infect Control Hosp Epidemiol. 2014, 35, 605.
5. Mehdizadeh, M.; Yang, J. Macromol. Biosci. 2013, 13, 271.
6. Artzi, N.; Zeiger, A. Boehning, F.; bon Ramos, A.; Vliet, K. V.; Edelman, E. R. ActaBiomater. 2011, 7, 67.
7. Lee, H.; Lee, B. P.; Messersmith, P. B. Nature 2007, 448, 338.
8. Mahdavi, A.; Ferreira, L.; Sundback, C.; Nicho, J. W.; Chan, E. P.; Carter, D. J. D.; Bettinger, C. J.; Patanavanich, S.; Chignozha, L.; Ben-Joseph, E.; Galakatos, A.; Pryor, H.; Pomerantseva, I.; Masiakos, P. T.; Faquin, W.; Zumbuehl, A.; Hong, S.; Borenstei, J.; Vacanti, J.; Langer, R.; Karp, J. M. Proc. Natl. Acad. Sci. USA 2008, 105, 2307. 9. Wang, D.-A.; Varghese, S.; Sharma, B.; Strehin, I; Fermanian, S.; Gorham, J.; Fairbrother, D. H.; Cascio, B.; Elisseeff, J. H. Nat. Mater. 2007, 6, 385.
10. Yang, S. Y. O'Cearbhaill, E. D.; Sisk, G. C.; Park, K. M.; Cho, W. K.; Villiger, M.; Bouma, B. E.; Pomahac, B.; Karp, J. M. Nat. Commun. 2013, 4, 1702.
11. Radosevich, M.; Goubran, H. A.; Burnouf, T. Vox Sang. 1997, 72, 133.
12. Spotnitz, W. D. World J. Surg. 2010, 34, 632. 13. Chenite, A.; Hoemann, C.; Buschmann, M.; Sereqi, A.; Sun, J. WO 2004006961 A1 14. Athanasiadis, T.; Hanton, L. R.; Moratti, S. C.; Robinson, B. H.; Robinson, S. R.; Shi, Z.; Simpson, J. Wormald, P. J. WO 2009028965 A1
15. Aziz, M. A.; Cabral, J. D.; Brooks, H. J. L.; Moratti, S. C.; Hanton, L. R. Antimicrob. Agents Chemother. 2012, 56, 280.
16. Giano, M. C.; Ibrahim, Z.; Medinal, S. H.; Sarhane, K. A.; Christensen, J. M.; Yamada, Y.; Brandacher, G.; Schneider, J. P. Nat. Commun. 2014, 5, 4095.
17. Brunota, C.; Ponsonnetc, L.; Lagneaua, C.; Fargeb, C.; Picarte, C.; Grosgogeata, B.; Biomaterials 2007, 28, 632.
18. Moghimi, S. M.; Symonds, P.; Murray, J. C.; Hunter, A. C.; Debska, G.; Szewczyk, A. Mol. Ther. 2005, 11, 990.
19. Taubes, G. Science 2008, 321, 356.
20. Walsh, C. Nature 2000, 406, 775.
21. Alekshun, M. N.; Levy, S. B. Cell 2007, 128, 1037.

### SUMMARY

The present disclosure relates to a polymer network comprising a compound of Formula I

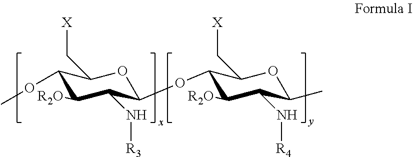

cross-linked to a compound selected from the group consisting of a compound of Formula II,

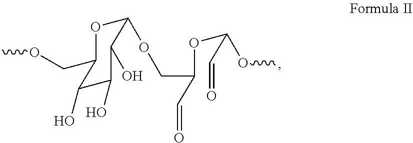

hyaluronate aldehyde, alginate aldehyde, dextran aldehyde, starch aldehyde, and chitosan aldehyde,
wherein,
X is selected from the group consisting of OR₁, and

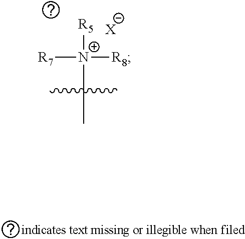

R₁ is selected from the group consisting of hydrogen, and

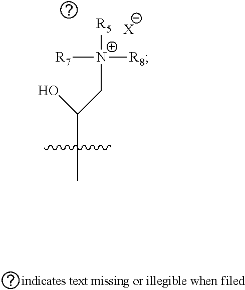

R₂ and R₄ are independently selected from the group consisting of hydrogen, and

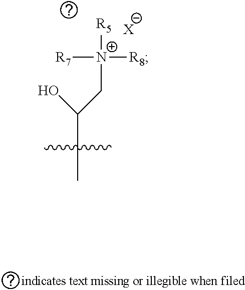

R₅, R₆, and R₇ are independently selected from the group consisting of C₁₋₁₂ alkyl, C₅₋₁₀ aryl, and

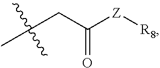

wherein alkyl, and aryl are optionally substituted with halogen, C₁₋₁₂ alkyl, and C₅₋₁₀aryl;
R₈ is selected from the group consisting of C₁₋₁₂ alkyl, and C₅₋₁₀ aryl wherein alkyl and aryl are optionally substituted with halogen, C₁₋₁₂ alkyl, and C₅₋₁₀ aryl, and Z is O or NH;
R₃ is selected from the group consisting of hydrogen, and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of each R₂ and R₄ with hydrogen, or

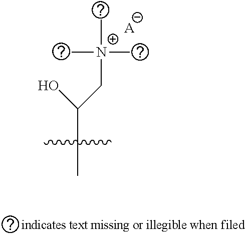

in the compound of Formula I is in the range of 20-100%; degree of substitution of R₃ with hydrogen or —COR₉ in the compound of Formula I is in the range of 20-100%.

The present disclosure further relates to a method of preparing the polymer network.

The present disclosure also relates to a composition comprising a polymer network as mentioned above and to a method of preparing the composition.

The present disclosure further relates to an antimicrobial polymeric hydrogel comprising a polymer network as mentioned above and to a method of preparing the antimicrobial polymeric hydrogel.

The present disclosure further relates to a hydrogel having the polymer network, for use in antimicrobial injectable bio-adhesive.

The present disclosure further relates to use of hydrogel, in treating infection or condition in a patient, wherein said infection or condition is caused by a microorganism selected from the group consisting of bacteria, virus, fungi, and protozoa. The patient is a mammal.

The present disclosure further relates to a method of treating a disease or infection or condition in a patent, said method comprising administering to a patient the hydrogel comprising the polymer network as mentioned above, wherein said disease or infection or condition is caused by microorganism selected from the group consisting of bacteria, virus, fungi and protozoa.

The present disclosure further relates to a kit to obtain the polymer network.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 illustrates the conductivity values of cationic chitosan derivatives (a) HTCC 1; (b) HTCC 2; (c) HTCC 3; (d) HTCC 4; (e) HTCC 5; and (f) HTCC 6; as a function of AgNO₃ volume added, in accordance with an embodiment of the present disclosure.

FIG. 2 illustrates the antibacterial kinetics of quaternary chitosan derivatives against (a) S. aureus; and (b) E. coli respectively, in accordance with an embodiment of the present disclosure.

FIG. 3 shows the propensity of bacterial resistant development of HTCC polymer, in accordance with an embodiment of the present disclosure.

FIG. 4 shows antibacterial activity of the injectable hydrogel. Optical density values of hydrogel treated and non-treated bacterial suspension at 600 nm for (a) S. aureus; (b) E. coli; (c) P. aeruginosa; (d) MRSA; (e) VRE; and (f) K. pneumoniae respectively, in accordance with an embodiment of the present disclosure.

FIG. 5 illustrates the antibacterial activity of hydrogels with or without antibiotics. Optical density value of hydrogel-treated and non-treated bacterial suspension at 600 nm for (a) S. aureus with an initial bacterial count of 10⁷ CFU/mL; (b) E. coli with an initial bacterial count of 10⁷ CFU/mL. Optical density values of hydrogel-treated and non-treated bacterial suspension at 600 nm (c) MRSA with an initial bacterial count of 10⁸ CFU/mL treated with hydrogel loaded with vancomycin; (d) MRSA with an initial bacterial count of 10⁹ CFU/mL treated with hydrogel loaded with vancomycin, in accordance with an embodiment of the present disclosure.

FIG. 6 shows the release kinetics of the antibacterial hydrogel were HTCC is not leached from bioadhesive gels at 10⁴ CFU/mL (a) S. aureus and (b) E. coli exposed to cell culture inserts containing adhesive gels or soluble HTCC at the same concentrations utilized to form the hydrogels, (c) 10⁴ CFU/mL MRSA exposed to cell culture inserts containing adhesive gels loaded with antibiotic or soluble HTCC at the same concentrations utilized to form the hydrogels, in accordance with an embodiment of the present disclosure.

FIG. 7 shows the hemolytic activity of the antibacterial hydrogel: (a) Hemolytic activity of hydrogels as a function of HTCC wt % along with the control TCTP surface with and without Triton-X (TX). Phase-contrast images of hRBCs (b) on the control TCTP surface; on hydrogel surface of (c) 1% HTCC; (d) 1.5% HTCC; (d) 1.75% HTCC; (e) 2% HTCC; (f) 2.5% HTCC (g) on TCTP surface treated with Triton-X, in accordance with an embodiment of the present disclosure.

FIG. 8 shows in-vivo activity of the injectable hydrogel: (a) Survival curves for saline, adhesive only, cecal ligation and puncture (CLP) with application of hydrogel (2.5 wt % PDA cross-linked with 2.5 wt % HTCC 3 with or without vancomycin) and CLP only (n=8); (b) application of hydrogel to the punctured cecum during surgery: application area outlined with circle mark: Isolated punctured cecum 24 h after surgery; (c) control cecum, and (d) experimental cecum that received gel, in accordance with an embodiment of the present disclosure.

FIG. 9 illustrates the evaluation of hemostatic ability of the hydrogel: (a) control, (b) hydrogels, and (c) total blood loss from the damaged livers after 3 min, in accordance with an embodiment of the present disclosure.

FIG. 10. Wound healing ability of the injectable hydrogel: representative photographs of 18 mm diameter wounds excised on rats (a) without any hydrogel and (b) treated with the hydrogel, in accordance with an embodiment of the present disclosure.

FIG. 11. Antibacterial activity of the hydrogels. Bacterial count after 6 h when 150 μL of the pathogen was challenged against the hydrogel's surface: (a) S. aureus count with an initial amount 1.7×10⁵ CFU/mL (150 μL); (b) MRSA count with an initial amount 1.2×10⁴ CFU/mL; (c) S. aureus count with an initial amount 1.67×10⁷ CFU/mL and (d) MRSA count with an initial amount 1.1×10⁶ CFU/mL, in accordance with an embodiment of the present disclosure. Stars represent less than 50 CFU/mL.

FIG. 12. Inhibition of bacterial lawn growth induced by releasing the antibiotic (vancomycin) containing hydrogels. Each plate has a confluent lawn of MRSA cells where the antibiotic diffused out from the central hydrogel disc and killed the surrounding bacteria leaving a clear zone. The hydrogels consisted of PDA and HTCC containing (a) 2.5 wt % PDA with 0 wt % vancomycin and 2.0 wt % HTCC (IHV-0); (b) 2.5 wt % PDA with 0.05 wt % vancomycin and 2.0 wt % HTCC (IHV-1); (c) 2.5 wt % PDA with 0.3 wt % vancomycin and 2.0 wt % HTCC (IHV-2) and (d) 2.5 wt % PDA with 0.6 wt % vancomycin and 2.0 wt % HTCC (IHV-3). Activity due to release of vancomycin from the hydrogels against (e) S. aureus and (f) MRSA respectively, in accordance with an embodiment of the present disclosure.

FIG. 13. Antibiotic release from the vancomycin-containing hydrogels. The amount of antibiotic released at different time interval from (a) IHV-1; (b) IHV-2 and (c) IHV-3 respectively. IHV-1, IHV-2 and IHV-3 contained an initial 200 μg, 1200 μg and 2400 μg of vancomycin and were used for release kinetics by adding 1 mL of buffer solution at varying pH and replacing the old buffer with fresh one after every 24 h. The amount antibiotic content in the solution was then determined by UV-visible absorption spectroscopy. Cumulative release of vancomycin from (d) IHV-1; (e) IHV-2 and (f) IHV-3 respectively, in accordance with an embodiment of the present disclosure.

FIG. 14. In-vivo antibacterial efficacy with direct injection of bacteria. Gross internal anatomical images of mice injected subcutaneously with 10⁷ CFU/mL of MRSA (a) directly into the back; (b) into adhesive IHV-0 and (c) into adhesive IHV-2, all after 3 days. Blue circles indicate the site of application. Evaluation of antibacterial activity upon injection of MRSA subcutaneously in mice: (d) MRSA count after 72 h of infection at different conditions; p values (*) are 0.002, <0.0001 and <0.0001 for IHV-0, IHV-2 (same site) and IHV-2 (distal site) samples, in accordance with an embodiment of the present disclosure.

### DETAILED DESCRIPTION

In the structural formulae given herein and throughout the present disclosure, the following terms have been indicated meaning, unless specifically stated otherwise.

### Definitions

The term, “hydrogel” refers to a network of polymer chains that are water-insoluble. Hydrogels are super absorbent natural or synthetic polymers with a water content of over 90%. By virtue of their high water content, hydrogels exhibit the same degree of flexibility as a natural tissue.

The hydrogel compositions disclosed herein are biocompatible. The term “biocompatible” as used herein means that the said hydrogel compositions are non-toxic and do not cause irritation to the tissues in the vicinity, to an extent that the medical professional finds it safe to use the said hydrogel composition on the patient.

The term “buffer” refers to an acidic or basic aqueous solution, though the solution may or may not act as a buffer in the conventional sense, i.e., maintaining pH even after addition of an acid or a base in. The pH of the buffer solution that is used for each of the two (or more) composition components should be adjusted using routine optimization to achieve a final pH favorable to rapid gelation.

The terms “site of application” or like that represent the location where the two solutions come into contact with each other can refer to any location where it is desirable to form the hydrogels disclosed herein. In the context of treatment of patients after surgery, the “site of application” refers to the site of surgery where a surgical incision or cut has been made.

The term “effective amount” refers to the amount of composition required in order to obtain the effect desired. For example, a “bactericidal amount” of a composition refers to the amount needed in order to kill bacteria in a patient to a non-detectable degree. The actual amount that is determined to be an effective amount will vary depending on factors such as the size, condition, sex, and age of the patient and can be more readily determined by the caregiver.

The described hydrogels can be administered in various ways. They may be applied directly to the tissue or may be introduced into a patient by a laparoscopic or an arthroscopic way, depending on which part of the body the treatment is sought. The components may be mixed using a dual syringe spray tip applicator well known to those skilled in the art. However, in certain applications, a preferred way may be to use an air-assisted spray tip to make sure efficient mixing of components during application of the gel.

The term “alkyl” refers to a monoradical branched or unbranched saturated hydrocarbon chain having from 1 to 16 carbon atoms. This term is exemplified by groups such as methyl, ethyl, n-propyl, iso-propyl, n-butyl, iso-butyl, t-butyl, n-hexyl, n-decyl, tetradecyl, and the like. By way of further example, a C₁-C₁₆ alkyl contains at least one but no more than 16 carbon atoms. A methyl group (i.e., CH₃—) is an example of a C₁ alkyl radical. A dodecyl group (i.e., CH₃ (CH₂)₁₂—) is an example of a C₁₂ alkyl radical.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. The polymers described herein are not intended to be limited in any manner by the permissible substituents of organic compounds.

The term “substituted alkyl” refers to an alkyl group as defined above, having 1 to 10 substituents, selected from the group consisting of hydroxyl, alkyl, aryl, alkoxy, halogen, haloalkyl, perhaloalkyl, cyano, or keto.

“Halo” or “Halogen”, alone or in combination with any other term means halogens such as chloro (Cl), fluoro (F), bromo (Br), and iodo (I).

The term “aryl” refers to an aromatic carbocyclic group of 5 to 10 carbon atoms having a single ring or multiple rings, or multiple condensed (fused) rings.

The term “substituted aryl” refers to an aryl group as defined above having 1 to 4 substituents, selected from the group consisting of hydroxyl, alkyl, aryl, alkoxy, halogen, haloalkyl, perhaloalkyl, cyano, or keto.

The term “heteroaryl” refers to an aromatic cyclic group having 3 to 10 carbon atoms and having heteroatoms selected from oxygen, nitrogen and sulfur within at least one ring (if there is more than one ring). Such heteroaryl groups can have a single ring (e.g. pyridyl or furyl) or multiple condensed rings (e.g. indolizinyl, benzothiazolyl, or benzothienyl).

The term “TCTP” refers to tissue culture treated polystyrene plate.

The term “drug resistant bacterium” as used herein is a bacterium which is able to survive exposure to at least one drug. In some embodiments, the drug resistant bacterium is a bacterium which is able to survive exposure to a single drug or multiple drugs. Examples of drug resistant bacterium include but are not limited to vancomycin resistant bacterium or methicilin resistant bacterium.

As used herein, the term “microbicidal” means that the polymer produces a substantial reduction in the amount of active microbes present on the surface, preferably at least one log kill, preferably at least two log kill when an aqueous microbe suspension or an aerosol is applied at room temperature for a period of time, as demonstrated by the examples. In more preferred applications, there is at least a three log kill, most preferably a four log kill. Although 100% killing is typically desirable, it is generally not essential.

The present disclosure relates to the field of biotechnology and specifically to the development of novel biomaterials. More specifically the present invention relates to the formulations of injectable hydrogel which exhibits good bioadhesive properties and broad spectrum biocidal activity.

The present disclosure relates to a polymer network comprising two polymers.

The present disclosure provides a highly biocompatible and antimicrobial hydrogel that can be applied to a wound as bioadhesive to assist wound healing and prevent infections at the wound site and thus to provide the public with a useful choice.

The present disclosure further relates to development of a completely biocompatible antimicrobial injectable hydrogel capable of preventing infection itself as well as acts as bioadhesive.

The present disclosure further provides a composition comprising powerful antimicrobial injectable bioadhesive which delivers antibiotic locally and acts synergistically.

The present disclosure relates to a polymer network comprising a compound of Formula I

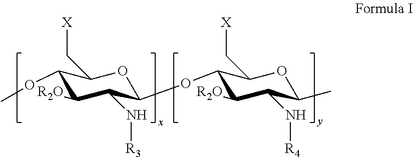

cross-linked to a compound selected from the group consisting of a compound of Formula II,

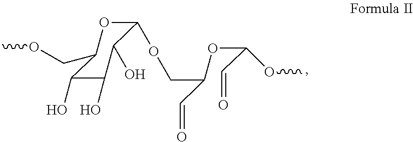

hyaluronate aldehyde, alginate aldehyde, dextran aldehyde, starch aldehyde, and chitosan aldehyde,
wherein,
X is selected from the group consisting of OR₁, and

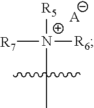

R₁ is selected from the group consisting of hydrogen, and

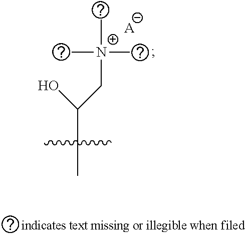

R₂ and R₄ are independently selected from the group consisting of hydrogen, and

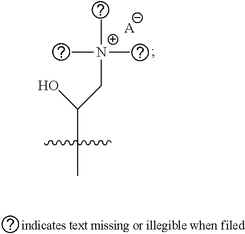

R₅, R₆, and R₇ are independently selected from the group consisting of C₁₋₁₂ alkyl, C₅₋₁₀aryl, and

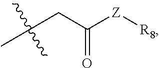

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and
aryl are optionally substituted with halogen, C₁₋₁₂ alkyl, and C₅₋₁₀ aryl, and Z is O or NH;
R₃ is selected from the group consisting of hydrogen and —COR₉;
R₉ is selected from the group consisting of C₁₋₁₆ alkyl, and C₅₋₁₀ aryl, wherein alkyl, and
aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of each R₂ and R₄ with hydrogen, or

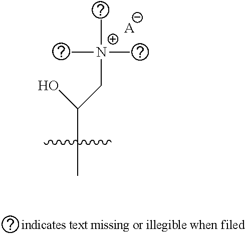

in the compound of Formula I is in the range of 20-100%;
degree of substitution of R₃ with hydrogen or —COR₉ in the compound of Formula I is in the range of 20-100%.

The present disclosure relates to a polymer network comprising a compound of Formula I

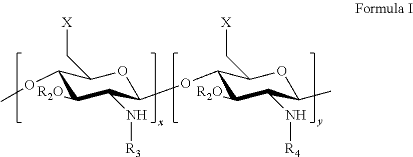

cross-linked to a compound of Formula II,

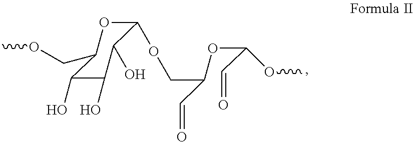

wherein,
X is selected from the group consisting of OR₁, and

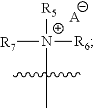

R₁ is selected from the group consisting of hydrogen, and

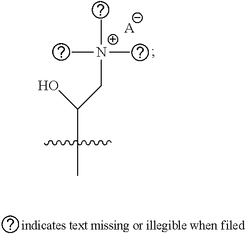

R₂ and R₄ are independently selected from the group consisting of hydrogen, and

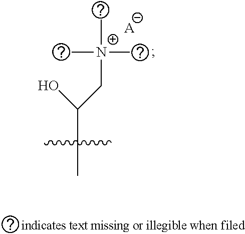

R₅, R₆, and R₇ are independently selected from the group consisting of C₁₋₁₂ alkyl, C₅₋₁₀ aryl, and

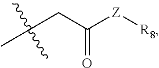

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is selected from the group consisting of hydrogen and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of each R₂ and R₄ with hydrogen, or

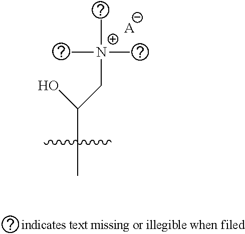

in the compound of Formula I is in the range of 20-100%;
degree of substitution of R₃ with hydrogen or —COR₉ in the compound of Formula I is in the range of 20-100%.

According to an embodiment, the present disclosure relates to a polymer network comprising a compound of Formula I

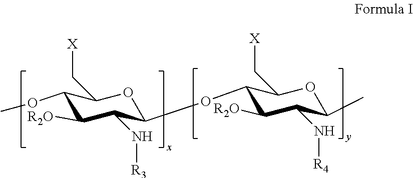

cross-linked to a compound selected from the group consisting of a compound of Formula II,

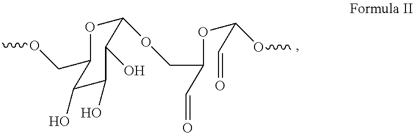

hyaluronate aldehyde, alginate aldehyde, dextran aldehyde, starch aldehyde, and chitosan aldehyde,
wherein;

### X is OR₁;

R₁ is selected from the group consisting of hydrogen, and

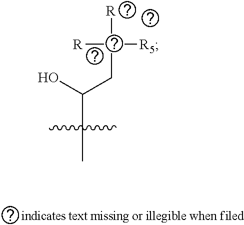

R₂ and R₄ are independently selected from the group consisting of hydrogen, and

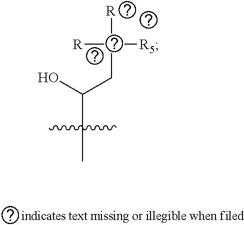

R₅, R₆, and R₇ are independently selected from the group consisting of C₁₋₁₂ alkyl, C₅₋₁₀aryl, and

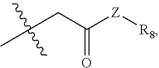

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl, wherein alkyl, and aryl are optionally substituted with halogen, C₁₋₁₂ alkyl, and C₅₋₁₀ aryl, and Z is O or NH;
R₃ is selected from the group consisting of hydrogen, and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of each R₂ and R₄ with hydrogen, or

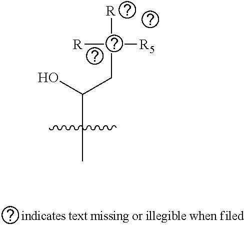

in the compound of Formula I is in the range of 20-100%;
degree of substitution of R₃ with hydrogen or —COR₉ in the compound of Formula I is in the range of 20-100%.

According to another embodiment, the present disclosure relates to a polymer network comprising a compound of Formula I

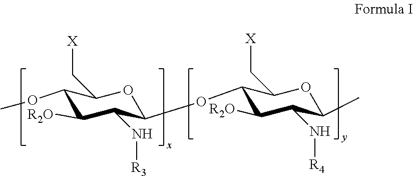

cross-linked to a compound selected from the group consisting of a compound of Formula II,

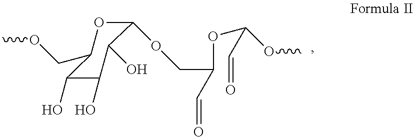

hyaluronate aldehyde, alginate aldehyde, dextran aldehyde, starch aldehyde, and chitosan aldehyde,
wherein;

### X is

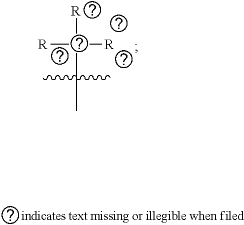

R₂ and R₄ are independently selected from the group consisting of hydrogen, and

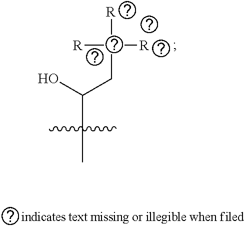

R₅, R₆, and R₇ are independently selected from the group consisting of C₁₋₁₂ alkyl, C₅₋₁₀ aryl, and

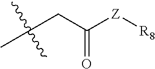

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is selected from the group consisting of hydrogen, and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of each R₂ and R₄ with hydrogen, or

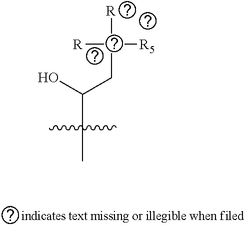

in the compound of Formula I is in the range of 20-100%;
degree of substitution of R₃ with hydrogen or —COR₉ in the compound of Formula I is in the range of 20-100%.

According to an embodiment, the present disclosure relates to a polymer network comprising a compound of Formula I

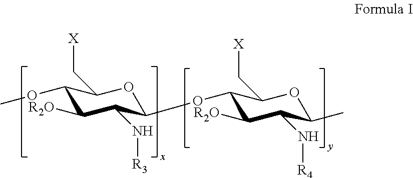

cross-linked to a compound selected from the group consisting of a compound of Formula II,

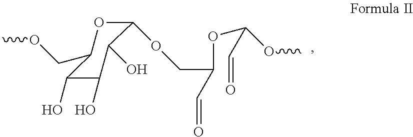

hyaluronate aldehyde, alginate aldehyde, dextran aldehyde, starch aldehyde, and chitosan aldehyde,
wherein;

### X is OR₁;

R₁ is selected from the group consisting of hydrogen, and

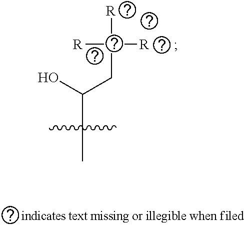

R₂ is selected from the group consisting of hydrogen, and

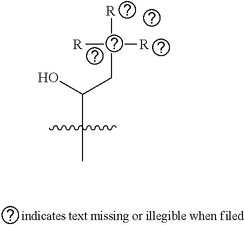

### R₄ is

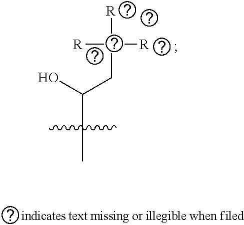

R₅, R₆ and R₇ are independently selected from the group consisting of C₁₋₁₂ alkyl, C₅₋₁₀ aryl, and

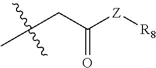

wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is selected from the group consisting of hydrogen, and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of R₂ with hydrogen or

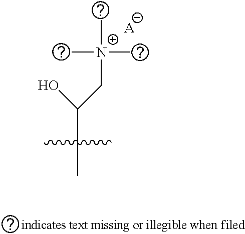

in the compound of Formula I is in the range of 20-100%;
degree of substitution of R₃ with hydrogen or —COR₉ in the compound of Formula I is in the range of 20-100%.
degree of substitution of R₄ with

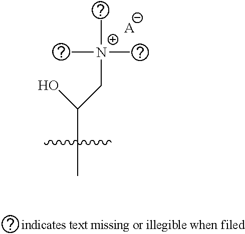

in the compound of Formula I is in the range of 20-100%.

According to another embodiment, the present disclosure relates to a polymer network comprising a compound of Formula I

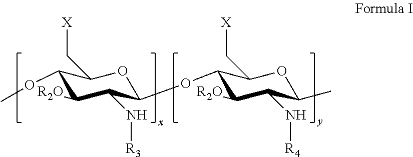

cross-linked to a compound selected from the group consisting of a compound of Formula II;

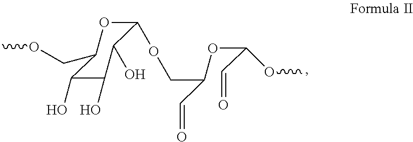

hyaluronate aldehyde, alginate aldehyde, dextran aldehyde, starch aldehyde, and chitosan aldehyde,
wherein;

### X is OR₁;

R₁ is selected from the group consisting of hydrogen, and

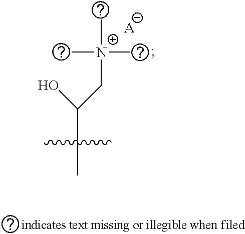

R₂ is hydrogen; R₄ is selected from the group consisting of hydrogen, and

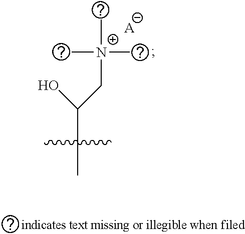

R₅, R₆ and R₇ are independently selected from the group consisting of C₁₋₁₂ alkyl, C₅₋₁₀ aryl, and

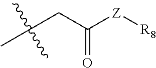

wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl and aryl are optionally substituted with halogen, C₁₋₁₂ alkyl, and C₅₋₁₀ aryl and Z is O or NH;
R₃ is selected from the group consisting of hydrogen, and —COR₉;
R₉ is selected from C₁₋₁₆ alkyl, and C₅₋₁₀ aryl, wherein alkyl and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of R₃ with hydrogen or —COR₉ in the compound of Formula I is in the range of 20-100%;
degree of substitution of R₄ with hydrogen or

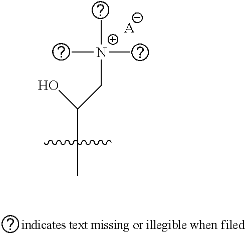

in the compound of Formula I is in the range of 20-100%.

According to an embodiment, the present disclosure relates to a polymer network comprising a compound of Formula I

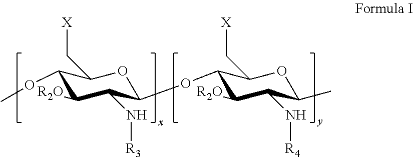

cross-linked to a compound selected from the group consisting of a compound of Formula II,

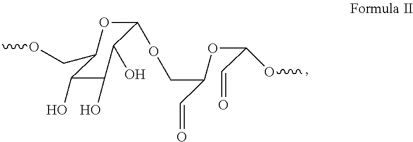

hyaluronate aldehyde, alginate aldehyde, dextran aldehyde, starch aldehyde, and chitosan aldehyde,
wherein;

### X is OR₁;

R₁ is hydrogen;
R₂ is hydrogen;

### R₄ is

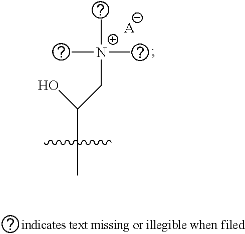

R₅, R₆ and R₇ are independently selected from the group consisting of C₁₋₁₂ alkyl, C₅₋₁₀ aryl, and

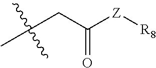

wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is hydrogen;
A^{⊖} is negatively charged counter anion;
x is 1 to 1000;
y is 1 to 1000;
wherein;
degree of substitution of R₄ with

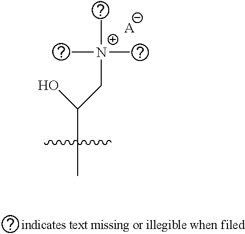

in the compound of Formula I is in the range of 20-100%.

According to another embodiment, the present disclosure relates to a polymer network comprising a compound of Formula I

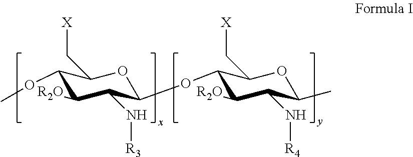

cross-linked to a compound selected from the group consisting of a compound of Formula II,

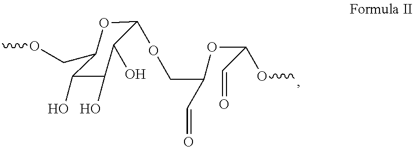

hyaluronate aldehyde, alginate aldehyde, dextran aldehyde, starch aldehyde, and chitosan aldehyde,
wherein;

### X is OR₁;

R₁ is hydrogen;
R₂ is hydrogen

### R₄ is

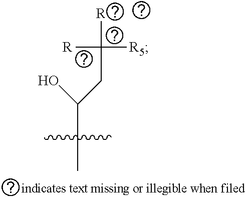

R₅, R₆ and R₇ are independently selected from the group consisting of C₁₋₁₂ alkyl, C₅₋₁₀ aryl, and

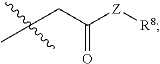

wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl and aryl are optionally substituted with halogen, C₁₋₁₂ alkyl, and C₅₋₁₀ aryl and Z is O or NH;
R₃ is —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of R₃ with —COR₉ in the compound of Formula I is in the range of 20-100%;
degree of substitution of R₄ with

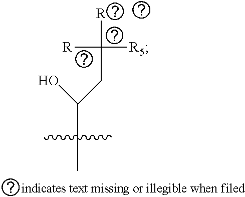

in the compound of Formula I is in the range of 20-100%.

According to still another embodiment, the present disclosure relates to a polymer network comprising a compound of Formula I

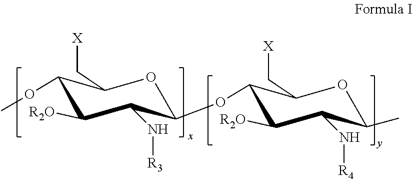

cross-linked to a compound selected from the group consisting of a compound of Formula II,

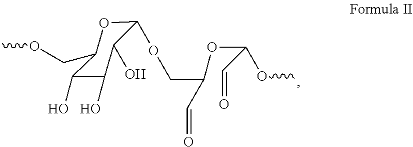

hyaluronate aldehyde, alginate aldehyde, dextran aldehyde, starch aldehyde, and chitosan aldehyde, wherein;

### X is

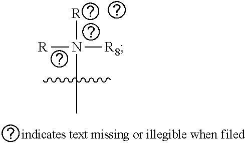

R₂ is hydrogen;
R₄ is selected from the group consisting of hydrogen, and

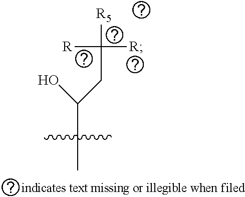

R₅, R₆ and R₇ are independently selected from the group consisting of C₁₋₁₂ alkyl, C₅₋₁₀ aryl, and

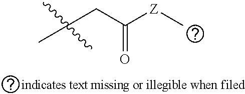

wherein alkyl and aryl are optionally substituted with halogen, C₁₋₁₂ alkyl, and C₅₋₁₀ aryl;
R₈ is selected from C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is selected from the group consisting of hydrogen and —COR₉;
R₉ is selected from the group consisting of C₁₋₁₂ alkyl, and C₆₋₁₀ aryl, wherein alkyl, and aryl are optionally substituted with halogen, C₁₋₁₂ alkyl, and C₆₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of R₃ with hydrogen or —COR₉ in the compound of Formula I is in the range of 20-100%;
degree of substitution of R₄ with hydrogen or

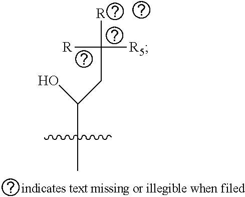

in the compound of Formula I is in the range of 20-100%.

According to another embodiment, the present disclosure relates to a polymer network comprising a compound of Formula I

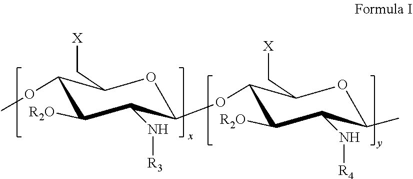

cross-linked to a compound selected from the group consisting of a compound of Formula II,

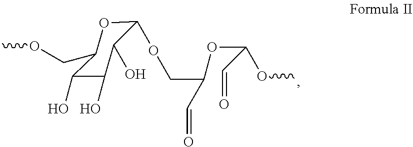

hyaluronate aldehyde, alginate aldehyde, dextran aldehyde, starch aldehyde, and chitosan aldehyde,
wherein;

### X is

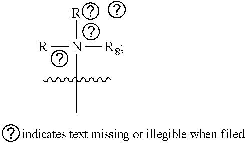

R₂ is selected from the group consisting of hydrogen, and

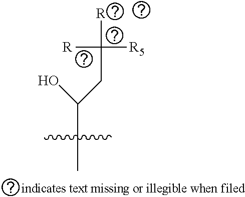

R₄ is hydrogen;
R₅, R₆, and R₇ are independently selected from the group consisting of C₁₋₁₂ alkyl, C₅₋₁₀ aryl, and

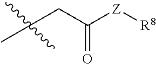

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is hydrogen;
A^{⊖} is negatively charged counter anion;
x is 1 to 1000;
y is 1 to 1000;
wherein;
degree of substitution of X in the compound of Formula I is in the range of 20-100%;
degree of substitution of R₂ with hydrogen or

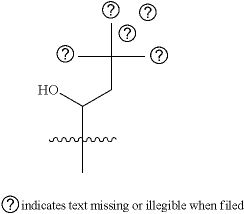 [SMILES: *C(O)CC(C)(C)C]

in the compound of Formula I is in the range of 20-100%.

According to an embodiment, the present disclosure relates to a polymer network comprising a compound of Formula I

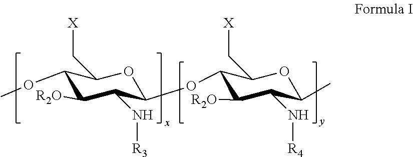

cross-linked to a compound selected from the group consisting of a compound of Formula II,

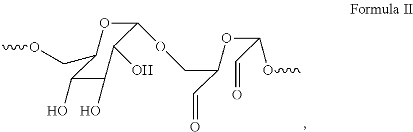

hyaluronate aldehyde, alginate aldehyde, dextran aldehyde, starch aldehyde, and chitosan aldehyde,
wherein;

### X is

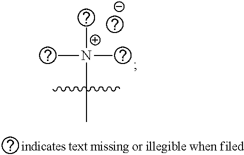

R₂ is hydrogen;

### R₄ is

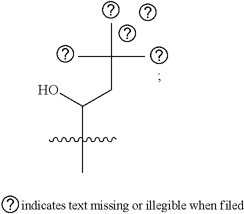 [SMILES: *C(O)CC(C)(C)C]

R₅, R₆ and R₇ are independently selected from the group consisting of C₁₋₁₂ alkyl, C₅₋₁₀ aryl, and

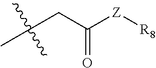

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is hydrogen;
A^{⊖} is negatively charged counter anion;
x is 1 to 1000;
y is 1 to 1000;
wherein;
degree of substitution of X in the compound of Formula I is in the range of 20-100%;
degree of substitution of R₄ with

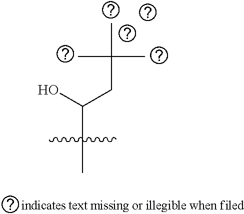 [SMILES: *C(O)CC(C)(C)C]

in the compound of Formula I is in the range of 20-100%.

In another embodiment, the present disclosure relates to a polymer network comprising a compound of Formula I

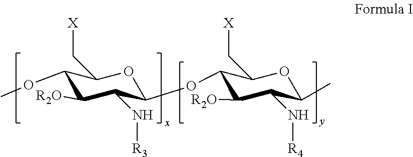

cross-linked to a compound selected from the group consisting of a compound of Formula II,

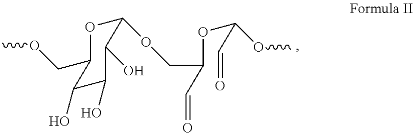

hyaluronate aldehyde, alginate aldehyde, dextran aldehyde, starch aldehyde, and chitosan aldehyde,
wherein;

### X is

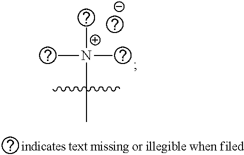

R₂ is hydrogen;

### R₄ is

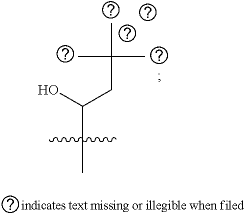 [SMILES: *C(O)CC(C)(C)C]

R₅, R₆ and R₇ are independently selected from the group consisting of C₁₋₁₂ alkyl, C₅₋₁₀ aryl, and

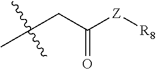

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of R₃ with —COR₉ in the compound of Formula I is in the range of 20-100%;
the degree of substitution of R₄ with

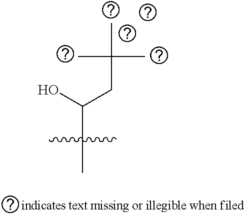 [SMILES: *C(O)CC(C)(C)C]

in the compound of Formula I is in the range of 20-100%.

According to another embodiment, the present disclosure relates to a polymer network comprising a compound of Formula I

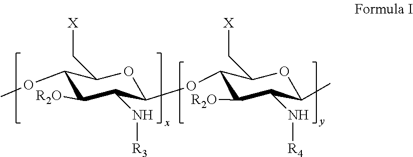

cross-linked to a compound selected from the group consisting of a compound of Formula II,

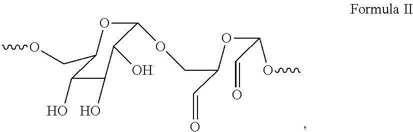

hyaluronate aldehyde, alginate aldehyde, dextran aldehyde, starch aldehyde, and chitosan aldehyde,
wherein;

### X is OR₁;

R₁ is hydrogen;
R₂ is hydrogen;

### R₄ is

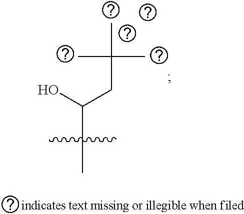 [SMILES: *C(O)CC(C)(C)C]

R₅, R₆, and R₇ are independently substituted with C₁₋₁₂ alkyl;
R₃ is hydrogen;
A^{⊖} is negatively charged counter anion;
x is 1 to 1000;
y is 1 to 1000;
wherein;
degree of substitution of R₄ with

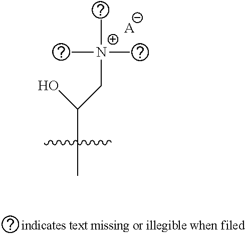

in the compound of Formula I is in the range of 20-100%.

According to another embodiment, the present disclosure relates to a polymer network comprising a compound of Formula I

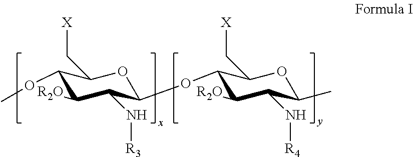

cross-linked to a compound selected from the group consisting of a compound of Formula II;

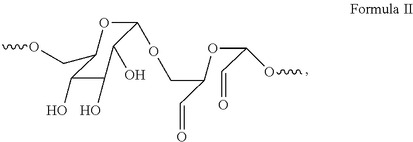

hyaluronate aldehyde, alginate aldehyde, dextran aldehyde, starch aldehyde, and chitosan aldehyde,
wherein;

### X is OR₁;

R₁ is hydrogen;
R₂ is hydrogen;

### R₄ is

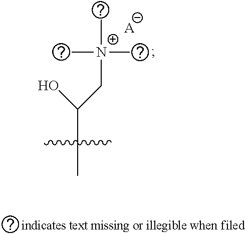

R₅, R₆, and R₇ are independently substituted with C₁₋₁₂ alkyl;
R₃ is —COR₉;
R₉ is C₁₋₁₆ alkyl;
A^{⊖} is negatively charged counter anion;
x is 1 to 1000;
y is 1 to 1000;
wherein;
degree of substitution of R₃ with —COR₉ in the compound of Formula I is in the range of 60-90%
degree of substitution of R₄ with

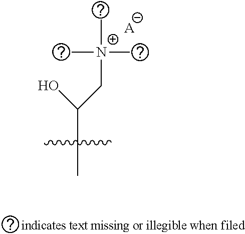

in the compound of Formula I is in the range of 20-100%.

According to another embodiment, the present disclosure relates to a polymer network comprising a compound of Formula I

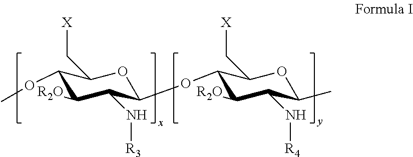

cross-linked to a compound selected from the group consisting of a compound of Formula II,

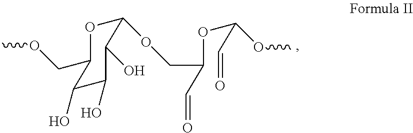

hyaluronate aldehyde, alginate aldehyde, dextran aldehyde, starch aldehyde, and chitosan aldehyde,
wherein;

### X is OR₁;

R₁ is hydrogen;
R₂ is hydrogen;

### R₄ is

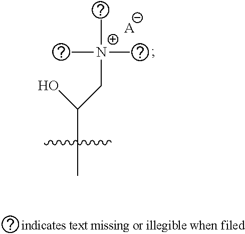

R₅, R₆, and R₇ are independently substituted with C₁ alkyl;
R₃ is hydrogen;
A^{⊖} is negatively charged counter anion;
x is 1 to 1000;
y is 1 to 1000;
degree of substitution of R₄ with

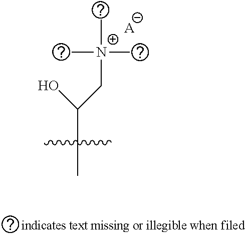

in the compound of Formula I is in the range of 20-100%.

According to another embodiment, the present disclosure relates to a polymer network comprising a compound of Formula I

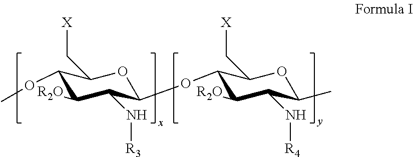

cross-linked to a compound of Formula II,

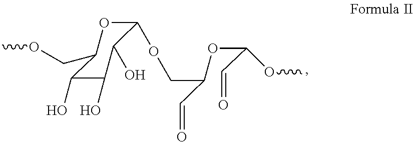

wherein;

### X is OR₁;

R₁ is hydrogen;
R₂ is hydrogen;

### R₄ is

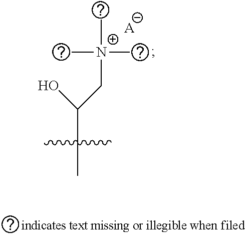

R₅, R₆, and R₇ are independently substituted with C₁ alkyl;
R₃ is —COR₉;
R₉ is C₁ alkyl;
A^{⊖} is negatively charged counter anion;
x is 1 to 1000;
y is 1 to 1000;
degree of substitution of R₃ with —COR₉ in the compound of Formula I is in the range of 60-90%.

According to yet another embodiment, the present disclosure relates to a polymer network wherein A^{⊖} is selected from the group consisting of Cl⁻, Br⁻, I⁻, OH⁻, HCO³⁻, CO₃²⁻, R₁₀COO⁻, R₁₀SO₄⁻, and R₁₀SO₃⁻, wherein R₁₀ is selected from the group consisting of hydrogen, C₁₋₆ alkyl, and C₅₋₁₀ aryl, wherein C₁₋₆ alkyl, and C₅₋₁₀ aryl are optionally substituted with hydroxyl, nitro, halogen, alkyl, aryl, or —COOR₁₀.

According to another embodiment, the present disclosure relates to a polymer network wherein the compound of Formula II is cross linked to the compound of Formula I through aldehyde group of Formula II and the amine group of Formula I.

According to yet another embodiment, the present disclosure relates to a polymer network wherein the compound of Formula I is N-(2-hydroxy)-propyl-3-trimethylammonium chitosan chloride.

According to yet another embodiment, the present disclosure relates to a process for the preparation of the polymer network comprising the step of cross linking the compound of Formula I

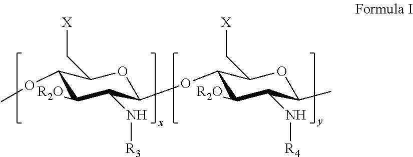

and a compound selected from the group consisting of a compound of Formula II,

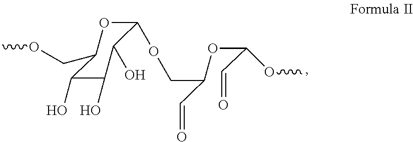

hyaluronate aldehyde, alginate aldehyde, dextran aldehyde, starch aldehyde, and chitosan aldehyde,
wherein,
X is selected from the group consisting of OR₁, and

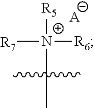

R₁ is selected from the group consisting of hydrogen, and

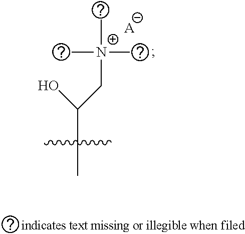

R₂ and R₄ are independently selected from the group consisting of hydrogen, and

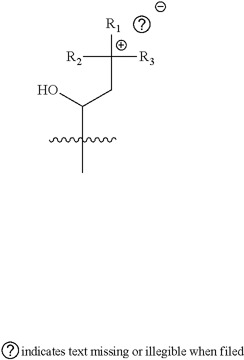

R₅, R₆, and R₇ are independently selected from the group consisting of C₁₋₁₂ alkyl, C₅₋₁₀ aryl, and

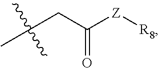

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is selected from the group consisting of hydrogen and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀aryl;
A^{⊖} is negatively charged counter anion;
x is 1 to 1000;
y is 1 to 1000;
wherein;
degree of substitution of X in the compound of Formula I is in the range of 20-100%;
degree of substitution of each R₂ and R₄ with hydrogen, or

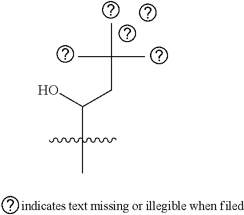 [SMILES: *C(O)CC(C)(C)C]

in the compound of Formula I is in the range of 20-100%;
degree of substitution of R₃ with hydrogen or —COR₉ in the compound of Formula I is in the range of 20-100%;
to obtain a polymer network.

The present disclosure more specifically relates to bioadhesive and antimicrobial injectable hydrogels based on quaternized chitosan derivative chemically cross-linked with polysaccharides having bisaldehyde functionality. The present disclosure relates to an injectable hydrogel which also serves as a local delivery vehicle to antibiotics.

According to yet another embodiment, the present disclosure relates to a polymer network as described herein, for use as antimicrobial infections.

According to an embodiment, the present disclosure relates to a polymer network as described herein, for use as antimicrobial agents in the treatment of diseases caused by bacteria, fungi, and virus.

According to another embodiment, the present disclosure relates to a polymer network as described herein, for use as antibacterial agents in the treatment of diseases caused by Gram-positive, Gram-negative bacteria or drug-resistant bacteria.

An embodiment of the present disclosure relates to a composition comprising a polymer network as described herein, in an aqueous solution.

According to another embodiment, the present disclosure relates to an antibacterial hydrogel comprising a polymer network consisting of N-(2-hydroxy)-propyl-3-trimethylammonium chitosan chloride (HTCC), and a second polymer polydextran aldehyde (PDA), wherein said polymer blend solidifies to form a solid hydrogel at physiological body temperature. In an another embodiment said polymer blend is formed by a (2-hydroxy)-propyl-3-trimethylammonium chitosan chloride (HTCC) or a compound of Formula I which is cross linked to a compound selected from the group consisting of a compound of Formula II, hyaluronate aldehyde, alginate aldehyde, dextran aldehyde, starch aldehyde, and chitosan aldehyde.

An embodiment of the present disclosure, relates to a composition comprising a polymer network as described herein, in an aqueous solution; wherein the polymer network comprises a compound of Formula I

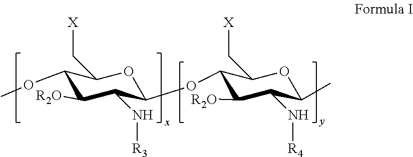

cross-linked to a compound of Formula II,

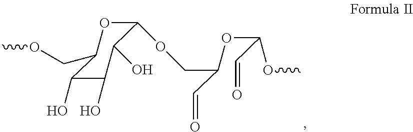

wherein;

### X is OR₁;

R₁ is hydrogen;
R₂ is hydrogen;

### R₄ is

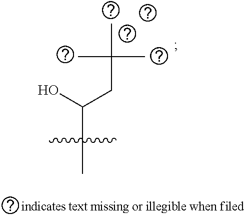 [SMILES: *C(O)CC(C)(C)C]

R₅, R₆, and R₇ are independently substituted with C₁ alkyl;
R₃ is —COR₉;
R₉ is C₁ alkyl;
A^{⊖} is negatively charged counter anion;
x is 1 to 1000;
y is 1 to 1000;
degree of substitution of R₃ with —COR₉ in the compound of Formula I is in the range of 60-90%.

According to an embodiment, the present disclosure relates to a composition, comprising the polymer network is in an aqueous buffered solution.

According to an embodiment, the present disclosure relates to a composition, wherein the buffer solution is selected from the group consisting of phosphate buffer and citrate buffer.

According to another embodiment, the present disclosure relates to a composition, wherein the compound of Formula I wt % is in the range of 0.5% to 15% w/w of the composition and the compound of Formula II wt % is in the range of 2% to 10% w/w of the composition.

In yet another embodiment, the present disclosure relates to a composition, wherein the compound of Formula I wt % is in the range of 0.5% to 2.5% w/w of the composition and the compound of Formula II wt % is in the range of 2% to 3% w/w of the composition.

In a further embodiment, the present disclosure relates to a composition, wherein the compound of Formula I wt % is in the range of 1% to 2.5% w/w of the composition and the compound of Formula II wt % is 2.5% w/w of the composition.

According to another embodiment, the present disclosure relates to a composition wherein the compound of Formula I wt % is 2.5% w/w of the composition and the compound of Formula II wt % is 2.5% w/w of the composition.

According to another embodiment, the present disclosure relates to a composition wherein the compound of Formula I is N-(2-hydroxy)-propyl-3-trimethylammonium chitosan chloride.

According to an embodiment, the present disclosure relates to a hydrogel comprising a polymer network and water, wherein the polymer network comprises a compound of Formula I

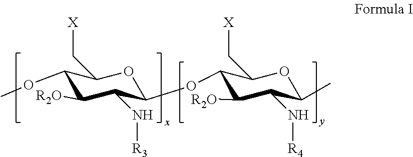

cross-linked to a compound selected from the group consisting of a compound of Formula II,

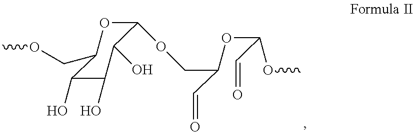

hyaluronate aldehyde, alginate aldehyde, dextran aldehyde, starch aldehyde, and chitosan aldehyde,
wherein,
X is selected from the group consisting of OR₁, and

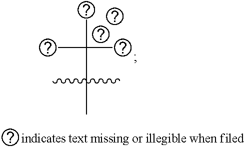 [SMILES: *C(C)(C)C]

R₁ is selected from the group consisting of hydrogen, and

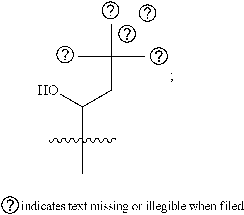 [SMILES: *C(O)CC(C)(C)C]

R₂ and R₄ are independently selected from the group consisting of hydrogen, and

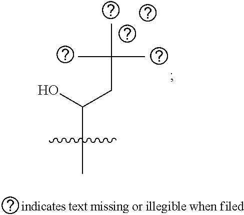 [SMILES: *C(O)CC(C)(C)C]

R₅, R₆, and R₇ are independently selected from the group consisting of C₁₋₁₂ alkyl, C₅₋₁₀ aryl, and

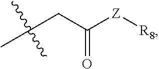

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is selected from the group consisting of hydrogen and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;

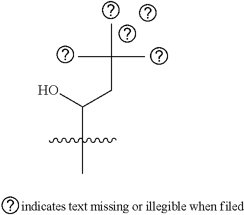 [SMILES: *C(O)CC(C)(C)C]

degree of substitution of each R₂ and R₄ with hydrogen, or in the compound of Formula I is in the range of 20-100%;
degree of substitution of R₃ with hydrogen or —COR₉ in the compound of Formula I is in the range of 20-100%.

An embodiment of the present disclosure relates to a hydrogel comprising a polymer network and water; wherein the polymer network comprises

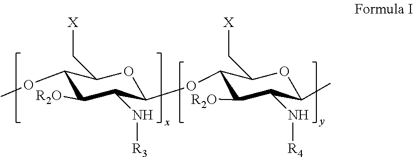

cross-linked to a compound of Formula II,

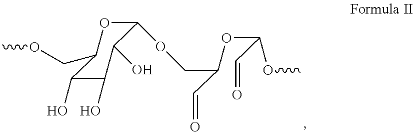

wherein;

### X is OR₁;

R₁ is hydrogen;
R₂ is hydrogen;

### R₄ is

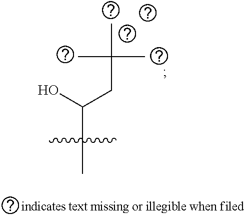 [SMILES: *C(O)CC(C)(C)C]

R₅, R₆, and R₇ are independently substituted with C₁ alkyl;
R₃ is —COR₉;
R₉ is C₁ alkyl;
A^{⊖} is negatively charged counter anion;
x is 1 to 1000;
y is 1 to 1000;
degree of substitution of R₃ with —COR₉ in the compound of Formula I is in the range of 60-90%.

According to an embodiment, the present disclosure relates to a hydrogel, wherein the compound of Formula I wt % is in the range of 2% to 15% w/w of the composition and the compound of Formula II wt % is in the range of 0.5% to 10% w/w of the composition.

According to an embodiment, the present disclosure relates to a hydrogel wherein, the compound of Formula I wt % is in the range of 2% to 3% w/w of the composition and the compound of Formula II wt % is in the range of 0.5% to 2.5% w/w of the composition.

According to another embodiment, the present disclosure relates to a hydrogel wherein, the compound of Formula I wt % is in the range of 1% to 2.5% w/w of the composition and the compound of Formula II wt % is 2.5% w/w of the composition.

According to yet another embodiment, the present disclosure relates to a hydrogel wherein, the compound of Formula I wt % is 2.5% w/w of the composition and the compound of Formula II wt % is 2.5% w/w of the composition.

According to another embodiment, the present disclosure relates to a hydrogel wherein, the compound of Formula I is N-(2-hydroxy)-propyl-3-trimethylammonium chitosan chloride.

In an embodiment, the hydrogel further comprises one or more biologically active agents.

According to another embodiment, the present disclosure relates to a hydrogel wherein, the biologically active agent is selected from antibiotics, silver nanoparticle, analgesic, anti-inflammatory drugs and growth factor such as human recombinant bone morphogenetic protein.

According to another embodiment, but not limited to, includes the antibiotics selected from the group of vancomycin, erythromycin, ciprofloxacin, colistin or antimicrobial peptides (AMP); analgesic like diclofenac Na salt, bupivacaine or any other local analgesic; anti-inflammatory drugs like aspirin, ibuprofen, naproxen sodium and growth factor like human recombinant bone morphogenetic protein (BMP). In one aspect, the compositions and method of the disclosure employ a BMP selected from BMP-2, BMP-3, BMP-4, BMP-5, BMP-6, or BMP-7.

According to another embodiment, the present disclosure relates to an antibacterial hydrogel comprising a polymer network comprising of a compound of Formula I

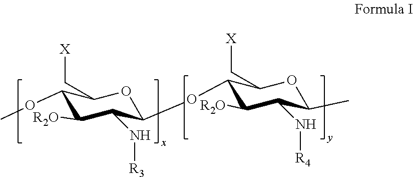

and a second polymer independently selected from the group consisting of a compound of Formula II,

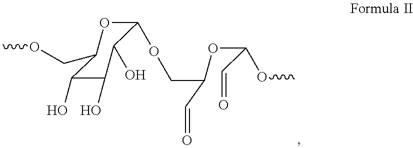

hyaluronate aldehyde, alginate aldehyde, dextran aldehyde, starch aldehyde, and chitosan aldehyde,
wherein,
X is selected from the group consisting of OR₁, and

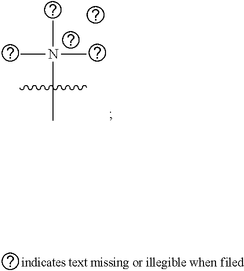

R₁ is selected from the group consisting of hydrogen, and

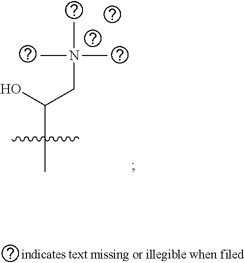

R₂ and R₄ are independently selected from the group consisting of hydrogen, and

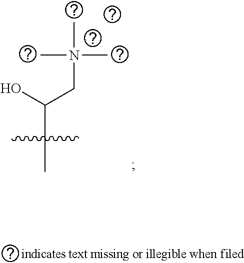

R₅, R₆, and R₇ are independently selected from the group consisting of C₁₋₁₂ alkyl, C₅₋₁₀ aryl, and

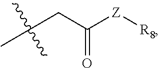

wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is selected from the group consisting of hydrogen and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of each R₂ and R₄ with hydrogen, or

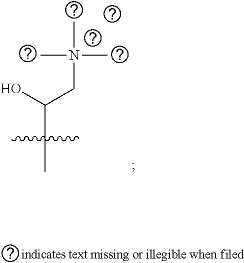

in the compound of Formula I is in the range of 20-100%;
degree of substitution of R₃ with hydrogen or —COR₉ in the compound of Formula I is in the range of 20-100%, along with the biologically active molecules wherein said polymer blend solidifies to form a solid hydrogel at physiological body temperature.

According to another embodiment, the present disclosure relates to an antibacterial hydrogel comprising a polymer network consisting of (2-hydroxy)-propyl-3-trimethylammonium chitosan chloride (HTCC), and a second polymer polydextran aldehyde (PDA), wherein said polymer blend solidifies to form a solid hydrogel at physiological body temperature, wherein said polymer blend is formed by a (2-hydroxy)-propyl-3-trimethylammonium chitosan chloride (HTCC) or a compound of Formula I which is cross linked to a compound selected from the group consisting of hyaluronate aldehyde, alginate aldehyde, dextran aldehyde, starch aldehyde, chitosan aldehyde and a compound of Formula II.

The present disclosure further relates to an antibacterial hydrogel with biologically active molecules comprising a polymer network consisting of (2-hydroxy)-propyl-3-trimethylammonium chitosan chloride (HTCC) or a compound of Formula I, and a second polymer polydextran aldehyde (PDA) or a compound of Formula II along with the effective amount of biologically active molecules wherein said polymer blend solidifies to form a solid hydrogel at physiological body temperature.

According to another embodiment, the present disclosure relates to an antibacterial hydrogel with silver nanoparticle comprising a polymer network consisting of (2-hydroxy)-propyl-3-trimethylammonium chitosan chloride (HTCC) or a compound of Formula I, and a second polymer polydextran aldehyde (PDA) or a compound of Formula II along with the preformed silver nanoparticle wherein said polymer blend solidifies to form a solid hydrogel at physiological body temperature.

The present disclosure relates to a process of preparing a hydrogel, the process comprising:

contacting a compound of Formula I,

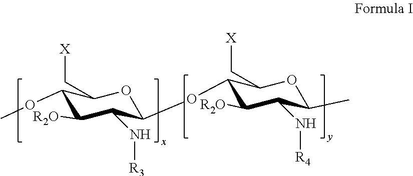

with the compound of Formula II;

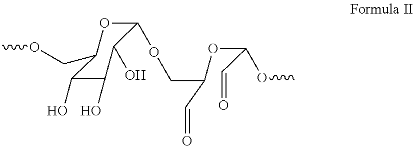

wherein;
X is selected from the group consisting of OR₁, and

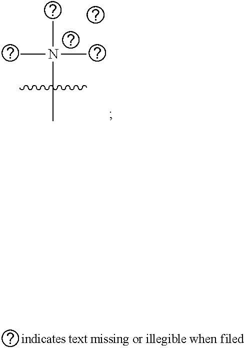

R₁ is selected from the group consisting of hydrogen, and

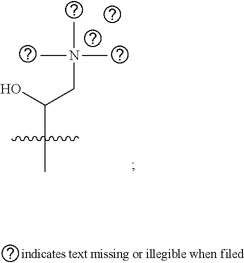

R₂ and R₄ are independently selected from the group consisting of hydrogen, and

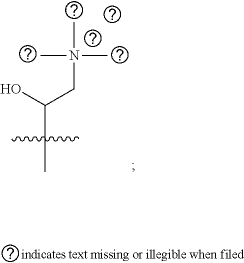

R₅, R₆ and R₇ are independently selected from the group consisting of C₁₋₁₂ alkyl, C₅₋₁₀aryl, and

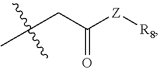

wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is selected from the group consisting of hydrogen, and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of each R₂ and R₄ with hydrogen, or

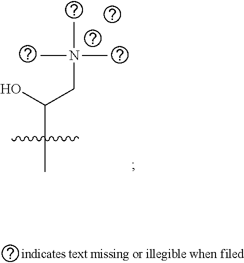

in the compound of Formula I is in the range of 20-100%;
degree of substitution of R₃ with hydrogen or —COR₉ in the compound of Formula I is in the range of 20-100%,
and water optionally in presence of a buffer to obtain the hydrogels;
wherein the Formula II and Formula I are present in an amount such that the ratio of RNH₂/RCHO group is between 0.5 to 1.5.

The buffer disclosed in the present disclosure is selected from the group consisting solutions of: citric acid, hydrochloric acid, phosphoric acid, sulfuric acid, AMPSO (3-[(1,1-dimethyl-2-hydroxyethyl)amino]2-hydroxy-propane-sulfonic acid), acetic acid, lactic acid, and combinations thereof. In certain embodiments, the acidic buffer solution is a solution of citric acid, hydrochloric acid, phosphoric acid, sulfuric acid, and combinations thereof. The buffer disclosed in the present disclosure is selected from the group consisting of phosphate or citrate buffer. In another embodiment, the buffer is phosphate buffer.

According to an embodiment, the present disclosure relates to the use of a polymer network, in the manufacture of a medicament as a hydrogel or composition for the treatment and/or prevention of diseases and/or disorders mediated by microbes.

According to an embodiment, the present disclosure relates to the use of a hydrogel or composition, for soft tissue repair.

According to an embodiment, the present disclosure relates to the use of a hydrogel or composition, for bone repair.

According to an embodiment, the present disclosure relates to the use of a hydrogel or composition, for repairing or resurfacing damaged cartilage.

According to an embodiment, the present disclosure relates to the use of a hydrogel or composition, in the manufacture of a medicament for soft tissue repair.

According to an embodiment, the present disclosure relates to the use of a hydrogel or composition, in the manufacture of a medicament for bone repair.

According to an embodiment, the present disclosure relates to the use of a hydrogel or composition, in the manufacture of a medicament for repairing or resurfacing damaged cartilage.

According to an embodiment, the present disclosure relates to the use of a hydrogel or composition, in the manufacture of a medicament for repairing meniscus.

According to an embodiment, the present disclosure relates to a method for treating a variety of diseases or conditions related to one or more microbial agents, comprising administering to a subject suffering from a condition mediated by one or more microbial agents a therapeutically effective amount of the hydrogel or composition.

According to an embodiment, the present disclosure relates to a method for repairing soft tissue, said method comprising the step of administering the hydrogel or the composition of the present disclosure at the site of a soft tissue in need of repair of a patient.

According to an embodiment, the present disclosure relates to a method for repairing or resurfacing a damaged cartilage, said method comprising the step of administering the hydrogel or the composition of the present disclosure in or around a cartilage in need of repair or resurfacing of a patient.

In one aspect of this embodiment, the present disclosure provides a kit comprising a compound of Formula I and a compound of Formula II and may or may not comprises a biologically active molecule; wherein each component is packaged separately and admixed immediately prior to use.

In another embodiment, the present disclosure relates to a kit wherein the compound of Formula I is contacted with the compound of Formula II to obtain the polymer network.

According to another embodiment, the present disclosure relates to a kit wherein either or both of (a) and (b) are provided in separate aqueous solutions optionally with a buffer.

According to another embodiment, the present disclosure relates to a kit wherein the aqueous solution of (a) is between 0.5% to 10% w/w and the aqueous solution of (b) is between 2% to 10% w/w.

According to yet another embodiment, the present disclosure relates to a kit wherein the kit further comprises an aqueous solution to allow cross linking of (a) and (b) to occur.

According to yet another embodiment, the present disclosure relates to a kit wherein the compound of Formula I is N-(2-hydroxy)-propyl-3-trimethylammonium chitosan chloride.

Thus, the present disclosure relates to the development of a novel injectable antimicrobial hydrogel from a biocompatible antibacterial polymer, (2-hydroxy)-propyl-3-trimethylammonium chitosan chloride (HTCC), and polydextran aldehyde (PDA). The present disclosure further relates to the formulations of non-toxic injectable antibacterial hydrogels using HTCC as antibacterial component.

The present disclosure further relates to the influence of HTCC content on the material's mechanical and biological properties affording an optimal formulation that sets at a rate conducive to surgical delivery. The hydrogel was found to be active against both drug-sensitive and drug-resistant Gram-positive and Gram-negative bacteria. The gel also acted as bioadhesive and prevented sepsis in murine model. Furthermore, antibiotics e.g., vancomycin was loaded into the hydrogel to develop even a more powerful antibacterial hydrogel which act synergistically against bacteria and delivers antibiotics locally.

Moreover, hydrogels with or without antibiotic were found to be non-toxic towards mammalian cells.

The examples given below are provided by the way of illustration only and therefore should not be construed to limit the scope of the invention.

### EXAMPLES

The disclosure is further illustrated by the following examples which in no way should be construed as being further limiting. One skilled in the art will readily appreciate that the specific methods and results described are merely illustrative.

Materials.

Chitosan with a degree of acetylation ˜85% (Mol. Wt. 15 kDa) was purchased from Polysciences, USA. Chitosan (Mol. Wt. 50-190 kDa), Dextran from Leuconostic spp. (Mr ˜40 kDa), glycidyltrimethylammonium chloride (GTMAC), acetic acid (AcOH), sodium periodate (NaIO₄), sodium nitrate, hydroxyl amine, and methyl orange, were purchased from Sigma-Aldrich, USA. Acetone, ethanol and other organic solvents were of analytical grade and purchased from SDFINE, India. The water used in all experiments was Millipore water with a resistivity of 18.2 MS cm. Bacterial strains S. aureus (MTCC 737), E. coli (MTCC 447) and A. baumannii (MTCC 1425) were purchased from MTCC (Chandigarh, India). Vancomycin-resistant Enterococcus faecium (VRE) (ATCC 51559), beta-lactum resistant Klebsilla pneumoniae (ATCC 700603), methicilin-resistant S. aureus (MRSA) (ATCC 33591) were obtained from ATCC (Rockville, Md.). All the clinical isolates were obtained from Department of Neuromicrobiology, National Institute of Mental Health and Neuro Sciences (NIMHANS), Bangalore, India. Bacterial growth media and agar were supplied by HIMEDIA, India. Nuclear magnetic resonance spectra (¹H NMR and ¹³C NMR) were recorded on a Bruker AMX-400 instrument (400 MHz) in deuterated solvents. FT-IR spectra of the solid compounds were recorded on Bruker IFS66 V/s spectrometer using KBr pellets. Elemental analysis was performed in a ThermoFinnigan FLASH EA 1112 CHNS analyzer. Eppendorf 5810R centrifuge was used for centrifugation. TECAN (Infinite series, M200 pro) Plate Reader was used to measure optical density. Studies on animal subjects were performed according to the protocols approved by Institutional Bio-safety Committee (IBSC) of Jawaharlal Nehru Centre for Advanced Scientific Research (JNCASR).

### Example 1

### Synthesis of Quaternary Chitosan Derivatives (HTCC):

Chitosan (2.5 g) was suspended in deionized water (200 mL), and then AcOH (1 mL, 0.5%, v/v) was added. The chitosan-AcOH mixture was stirred overnight at room temperature prior to the drop wise addition of GTMAC in three portions at two-hourly intervals. The mole ratio of GTMAC to chitosan was varied from 4:1 to 8:1 to produce quaternary chitosan derivatives with different degree of substitution (DS). After the final addition of GTMAC the reaction mixture was stirred at 55° C. for 18 h. Finally, the reaction mixture was diluted with 200 mL distilled water and the product was precipitated with excess of acetone (600 mL) with more than 90% yield.

TABLE 1
Reaction conditions for the synthesis of quaternized
chitosan derivatives and macromolecular characteristics

| | MW | GTMAC/ | Degree of | Solubility |
|---|---|---|---|---|
| | of chitosan | glucosamine | substitution | in |
| Sample | used (kDa) | molar ratio | (DS) (%) | water |
| HTCC 1 | 15 | 4:1 | 31 | + |
| HTCC 2 | 15 | 6:1 | 48 | + |
| HTCC 3 | 15 | 8:1 | 58 | + |
| HTCC 4 | 50-190 | 4:1 | 29 | + |
| HTCC 5 | 50-190 | 6:1 | 45 | + |
| HTCC 6 | 50-190 | 8:1 | 54 | + |

MW = molecular weight; GTMAC = glycidyltrimethylammonium chloride.

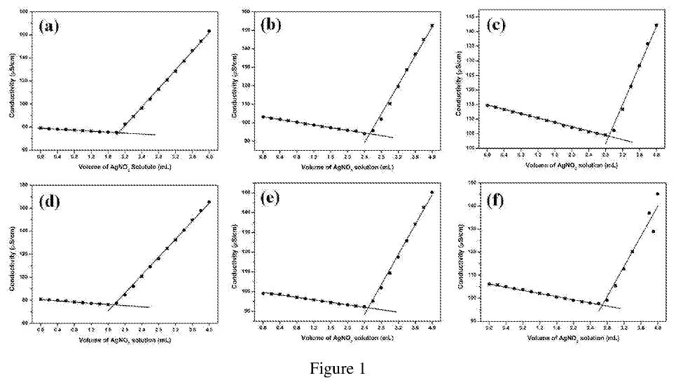

Such conditions not only favor the random substitution of the sugar units in the chitosan chain, but also selective grafting onto the primary amine groups. Introduction of quaternary ammonium groups onto chitosan as well as to ascertain the selective substitution of the primary amine groups was confirmed by ¹H NMR. The degree of substitution of the HTCC samples was derived by conductometric titration of Cl⁻ ions with AgNO₃ (FIG. 1). The characteristics of the HTCC samples are listed in Table 1. The degree of substitution in quaternary chitosan ranged from 29-58% thus giving a variety chitosan derivatives having different degree of quaternization.

### Example 2

### Antibacterial Activity of HTCC Polymers:

Antibacterial efficacy of HTCC polymers was assayed in a micro-dilution broth method. The 6 h grown culture gave ˜10⁹ CFU/mL of bacteria determined by spread plating method. The bacterial cultures were then diluted to give ˜10⁵ CFU/mL in nutrient media which were then used for determining antibacterial activity. Stock solutions were prepared by serial dilution of all the polymers using sterilized Milli-Q water. These dilutions (50 μL) were added to the wells of 96 well plate followed by the addition of about 150 μL of bacterial suspension (˜10⁵ CFU/mL). The plates were then incubated at 37° C. for 24 h. After the incubation, the optical density (OD) of the bacterial suspension was recorded using TECAN (Infinite series, M200 pro) Plate Reader at 600 nm. Each concentration was added in triplicate and the whole experiment was repeated at least twice. Finally, the antibacterial efficacy was determined by taking the average of triplicate OD values for each concentration and plotting it against concentration. The data was then subjected to sigmoidal fitting and from the curve the antibacterial activity was determined as the point where the OD value was similar to that of control having no bacteria. The antibacterial activity was thus expressed as minimum inhibitory concentration (MIC).

TABLE 2
Minimum inhibitory concentration and
hemolytic activity of HTCC polymers

| | MIC (μg/mL) | | | | | | |
|---|---|---|---|---|---|---|---|
| | Drug- | | | | | | |
| | sensitive bacteria | | | Drug-resistant bacteria | | | HC₅₀ |
| | S. | E. | P. | | | K. | (μg/ |
| Polymer | aureus | coli | aeruginosa | MRSA | VRE | pneumoniae | mL) |
| HTCC 1 | 250 | 500 | 500 | 250 | 250 | 500 | >12000 |
| HTCC 2 | 250 | 500 | 500 | 250 | 250 | 500 | >12000 |
| HTCC 3 | 125 | 250 | 250 | 125 | 125 | 250 | >12000 |
| HTCC 4 | 250 | 500 | 500 | 250 | 250 | 500 | >12000 |
| HTCC 5 | 250 | 250 | 500 | 250 | 250 | 500 | >12000 |
| HTCC 6 | 125 | 250 | 250 | 125 | 125 | 250 | >12000 |

MIC = minimum inhibitory concentration;
MRSA = Methicillin-resistant S. aureus;
VRE = vancomycin-resistant E. faecium;
HC₅₀ = hemolytic concentration at which 50% hemolysis occurs

All the quaternary chitosan derivatives showed antibacterial activity against all the bacteria tested (Table 2). The polymer with highest degree of substitution (hence highest degree of quaternization) showed maximum activity against both drug-sensitive and drug-resistant bacteria, e.g., HTCC 3 showed MIC values 125-250 μg/mL against Gram-positive bacteria whereas 250-500 μg/mL against Gram-negative bacteria. Thus HTCC 3 was successively used in hydrogel formulation. The quaternary chitosan derivatives were also found to be active against various multi-drug resistant clinical isolates (Table 3). HTCC 3 was again found to be the most active polymer against all 12 clinical isolates tested. This further showed that polymer HTCC 3 would be a potent antibacterial component in developing two components based injectable antibacterial hydrogel.

TABLE 3
Antibacterial activity of chitosan derivatives against clinical isolates

| | MIC (μg/mL) | | | | | |
|---|---|---|---|---|---|---|
| Bacterial | HTCC | HTCC | HTCC | HTCC | HTCC | HTCC |
| clinical isolates | 1 | 2 | 3 | 4 | 5 | 6 |
| MRSA R3545 | 125 | 125 | 62.5 | 125 | 125 | 62.5 |
| MDR | | | | | | |
| MRSA R3889 | >250 | 250 | 250 | >250 | 250 | 250 |
| MDR | | | | | | |
| E. coli R3597 | 250 | 125 | 125 | 250 | 125 | 125 |
| MDR | | | | | | |
| E. coli R250 | >250 | 250 | 250 | 250 | 250 | 250 |
| MDR | | | | | | |
| A. baumannii | 250 | 125 | 125 | 250 | 125 | 125 |
| R676 NDM-1 | | | | | | |
| A. baumannii | >250 | >250 | 250 | >250 | >250 | 250 |
| R674 | | | | | | |
| E. cloacae R3921 | 125 | 62.5 | 62.5 | 125 | 62.5 | 62.5 |
| NDM-1 | | | | | | |
| E. cloacae R2928 | >250 | 250 | 125 | >250 | 250 | 125 |
| K. pneumonia | 125 | 125 | 62.5 | 125 | 62.5 | 62.5 |
| R3421 MDR | | | | | | |
| K. pneumonia | >250 | >250 | 250 | >250 | 250 | 250 |
| R3949 NDM1 | | | | | | |
| P. aeruginosa | >250 | >250 | >250 | >250 | >250 | >250 |
| R596 MDR | | | | | | |
| P. aeruginosa | >250 | 250 | 250 | >250 | 250 | 250 |
| R590 MDR | | | | | | |

MIC = minimum inhibitory concentration;
MRSA = Methicilin-resistant S. aureus;
VRE = vancomycin-resistant E. faecium;
MDR = multi drug-resistance;
NDM = New Delhi Metallo beta lactamase

### Example 3

### Kinetics of Antibacterial Activity of HTCC Polymers:

The rate at which the polymers killed bacteria was evaluated by performing time kill kinetics. Briefly, bacteria (S. aureus and E. coli) were grown in suitable growth medium at 37° C. for 6 h. Two most active polymers (HTCC 3 and HTCC 6) were added to the bacterial suspension (150 μL of S. aureus of approximately 4.75×10⁴ CFU/mL and E. coli of approximately 5.58×10⁴ CFU/mL) in 96-well plate each polymer at two different concentrations (MIC and 6×MIC, 50 μL). The plate was then incubated at 37° C. At different time intervals (0, 30, 60, 90, 120, 240 and 360 min), 10 μL of the aliquots from the bacterial suspension was withdrawn and diluted serially (10-fold serial dilution) in 0.9% saline. 20 μL of the dilution was plated on solid agar plates and incubated at 37° C. for 24 h. The bacterial colonies were counted and results are represented in logarithmic scale, i.e. log₁₀ (CFU/mL). A similar experiment was performed by using water (50 μL) as control.

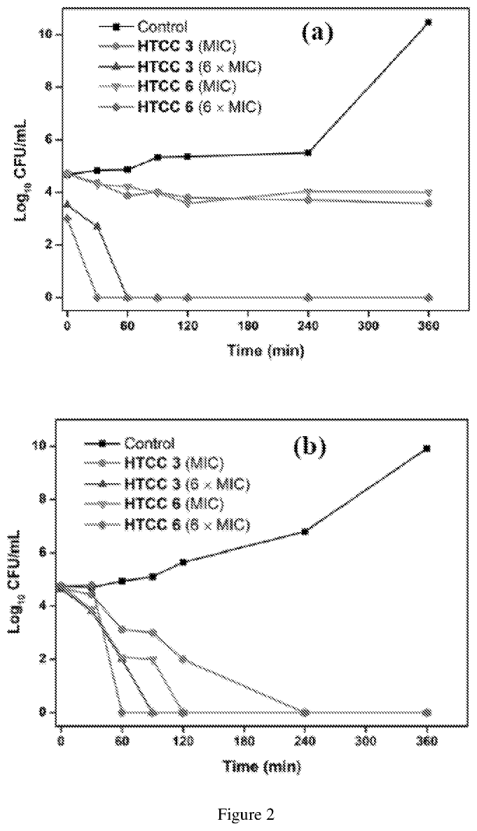

The polymers showed rapid killing of bacteria as it killed both Gram-positive and Gram-negative bacteria within 60-90 minutes at 6×MIC. At minimum inhibitory concentration, the polymers showed bacteriostatic effect against S. aureus whereas showed bactericidal effect against E. coli (FIG. 2).

### Example 4

### Resistance Studies:

One of the most active polymers HTCC 3 was used to evaluate the propensity of developing bacterial resistance towards the polymers. First MIC of HTCC 3 was determined against both Gram-positive and Gram-negative S. aureus and A. baumannii in a way as described in antibacterial assay and subsequently the polymer was challenged repeatedly at the sub-MIC (MIC/2) level. Two control antibiotics norfloxacin and colistin were chosen for S. aureus and for A. baumannii, respectively. In case of norfloxacin and colistin also, the initial MIC values were determined against respective bacteria. After the initial MIC experiment, serial passaging was initiated by transferring bacterial suspension grown at the sub-MIC of the polymer/antibiotics and was subjected to another MIC assay. After 24 h incubation period, cells grown at the sub-MIC of the test compound/antibiotics were once again transferred and assayed for MIC experiment. The process was repeated for 14 passages for both S. aureus and A. baumannii respectively. The fold increase in MIC for test polymer to the control antibiotics was plotted against the number of passages to determine the propensity of bacterial resistance development.

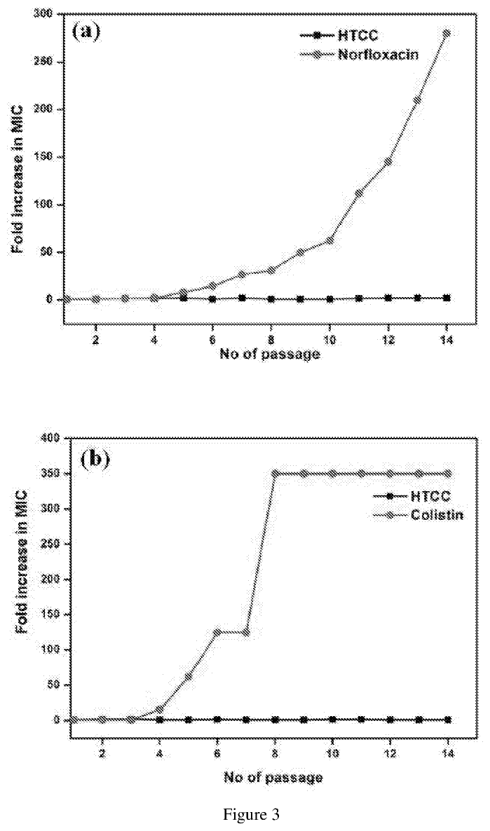

The cationic polymer showed no change in MIC against both the bacteria even after 14 passages whereas gradual increase in MIC was observed for norfloxacin against S. aureus and colistin against A. baumannii respectively (FIG. 3). The above results thus indicated that bacteria were less prone to develop resistance against this type of polymer making it an ideal candidate in developing antibacterial injectable hydrogel.

### Example 5

### Hemolytic Activity of HTCC Polymers:

Studies on human subjects were performed according to the protocols approved by Institutional Bio-Safety Committee (IBSC) of Jawaharlal Nehru Centre for Advanced Scientific Research (JNCASR). Red blood cells (RBCs) were isolated from freshly drawn, heparinised human blood and resuspended in 1×PBS (5 vol %). RBC suspension (150 μL) was then added to solutions of serially diluted polymers in a 96-well plate (50 μL). Two controls were prepared, one without the compounds and the other with 50 μL of 0.1 vol % solution of Triton X-100. The plate was then incubated for 1 h at 37° C. After the incubation, the plate was centrifuged at 3500 rpm for 5 minutes. Supernatant (100 μL) from each well was then transferred to a fresh 96-well plate and absorbance at 540 nm was measured. Percentage of hemolysis was determined as (A−Aₒ)/(Aₜₒₜₐₗ−Aₒ)×100, where A is the absorbance of the test well, Aₒ is the absorbance of the negative control (the wells having no compound), and Aₜₒₜₐₗ the absorbance of completely lysed cells (wells with Triton X-100), all at 540 nm. To visualize the effect of cationic polymers, treated and non-treated RBC were also imaged by optical microscopy.

The polymers showed no detectable hemolysis even at 6000 μg/mL. Only 2-5% hemolysis observed at 12000 μg/mL. Thus, HC₅₀ values were found to be very high for these polymers making these polymers highly compatible towards mammalian cells (Table 1).

### Example 6

### In-Vivo Systemic Toxicity of HTCC Polymers:

Female BALB/c mice (6-8 weeks, 18-22 g) were used for systemic toxicity studies. Mice were put into control and test groups with 5 mice per group. Control groups received 200 μL of sterilized saline. Different doses (5.5, 17.5, 55 and 175 mg/kg) of the test drugs were used as per the OECD guidelines. Polymer solution (200 μL) in sterilized saline was injected into each mouse (5 mice per group) through intraperitoneal (i.p.) and subcutaneous (s.c.) route of administration. All the mice were monitored for the next 14 days after the treatment. During the observation period of 14 days, no onset of abnormality was found even in the high dose group (175 mg/kg). The 50% lethal dose (LD₅₀) was estimated according to the up-and-down method. For acute dermal toxicity studies, back of the mice was shaved 24 h before the experiment. To the shaved region the polymer solution (200 μL) of different concentration was applied. Adverse effect on the skin of mice was monitored along with mortality rate for 14 days post treatment. Studies on animals were performed in accordance with protocols approved by the Institutional Animal Ethics Committee (IAEC) at the Jawaharlal Nehru Centre for Advanced Scientific Research (JNCASR).

TABLE 4
In-vivo systemic toxicity of the HTCC polymers

| | LD₅₀ (mg/kg) | | |
|---|---|---|---|
| | Intra- | Sub- | Acute |
| | peritoneal | cutaneous | dermal |
| Polymer | (i.p.) | (s.c.) | toxicity (d.t.) |
| HTCC 3 | >175 | >175 | >200 |

The cationic chitosan derivative (HTCC 3) showed very high LD50 values in all three routes of administrations. HTCC 3 showed LD₅₀ values of >175 mg/kg in i.p., s.c. administration and in acute dermal toxicity experiments. Thus, the polymer was found to be highly non-toxic under in-vivo conditions.

### Example 7

### In-Vivo Sub-Chronic Toxicity Studies of HTCC Polymers:

Female BALB/c mice (6-8 weeks, 18-22 g) were used for both acute and sub-chronic toxicity studies (four groups of mice, 10 mice in each group). Polymer solution in sterilized saline (200 μL) was via given intra-peritoneal (i.p.) injection of HTCC 3 at a dosage of 55 mg/kg in two groups and the remaining two groups were used as control groups. After 48 h, blood was collected from 20 mice (10 mice for HTCC 3, 10 mice for control) and analyzed for different parameters like alkaline phosphatase (ALP), creatinine, blood urea nitrogen, and electrolytes like sodium, potassium ions and chloride. Also, after 14 days, blood was collected from the remaining mice and analyzed for the abovementioned parameters.

TABLE 5
Clinical biochemistry parameters of HTCC polymers

| | | Treatment | | | |
|---|---|---|---|---|---|
| Clinical | | | HTCC 3 | HTCC 3 | Laboratory |
| biochemistry parameters | | Saline | (at day 2) | (at day 14) | range |
| Liver | ALT (IU/L) | 62.4 ± 19.1 | 56.3 ± 25.2 | 77.8 ± 29.1 | 50 ± 27 |
| functions | AST (IU/L) | 80.9 ± 18.7 | 87.6 ± 15.3 | 101.4 ± 23.2 | 100 ± 50 |
| Kidney | Creatinine(mg/dL) | 0.23 ± 0.07 | 0.23 ± 0.07 | 0.41 ± 0.13 | 0.38 ± 0.12 |
| Function | Urea nitrogen (mg/dL) | 18.4 ± 3.4 | 21.5 ± 2.9 | 20.3 ± 5.8 | 16 ± 7.2 |
| Electrolyte | Sodium ion (mg/dL) | 143 ± 1.6 | 147.4 ± 1.01 | 151.3 ± 1.64 | 152.3 ± 17 |
| Balance | Potassium ion (mg/dL) | 7.2 ± 0.7 | 5.7 ± 0.7 | 6.7 ± 0.3 | 8.9 ± 1.5 |
| | Chloride ion (mg/dL) | 111.5 ± 1.8 | 110.8 ± 2.2 | 108.9 ± 1.5 | 119.3 ± 13.5 |

ALT = Alanine transaminase; AST = aspartate transaminase

The sub-chronic toxicity to major organs in mice was evaluated by determining the clinical biochemistry parameters in the blood after a single-dose i.p. administration of HTCC 3 (at a dosage of 22.5 mg/kg). The derivative did not induce any adverse toxicity to major organs like liver and kidney and did not interfere with the balance of electrolytes in the blood of mice 2 days and 14 days post treatment compared to vehicle control and laboratory parameters (Table 5). The data are expressed as mean±standard deviation, based on values obtained from 10 mice (n=10 for the data from this report).

### Example 8

### Preparation Polydextran Aldehyde (PDA):

Dextran (40 kDa, 10 g, 0.06 mol glucose monomer) was dissolved in 400 mL of milli-Q water. Then of sodium periodate (9.91 g, 0.04 mol) dissolved in 100 mL of milli-Q water was added to the dextran solution and stirred for 24 h at room temperature in dark condition. After the reaction, the mixture was extensively dialyzed (MW cutoff-10 kDa) against milli-Q water over 3 days with frequent water changes. The oxidized dextran was then obtained after lyophilisation of the solution as white fluffy powder. Oxidation of dextran leads to the formation of multiple aldehyde species as well as the formation of various hemiacetals within the polymer. All of these species are in equilibrium when PDA is dissolved in aqueous solution. The relative oxidation, or percent functionalization, was determined by ¹³C NMR according to:

% of Functionality=(molGUi−molGU_{f}/molGUi)×100%

Here the difference with respect to the number of moles of glucose units before (molGUi) and after oxidation (molGU_{f}) is used to determine percent functionality. The initial number of moles of glucose units is known and represents the moles of glucose units available before oxidation. The moles of glucose units remaining after oxidation in the PDA was determined by NMR by integrating carbons 2 and 3 of the glucose ring, which had well-resolved chemical shifts. The molecular weight (Mw) of PDA was determined by gel permeation chromatography and was found to be 35 kDa (polydispersity index, PDI=1.3). The % of aldehyde functionality in PDA was found to be 39% (bisaldehyde group).

### Example 9

### Preparation and Characterization of the Hydrogels:

Hydrogels were prepared by first dissolving 50 mg of PDA (39% functionalized) in 1 mL of phosphate buffer (23.5 mM NaH₂PO₄, 80.6 mM Na₂HPO₄) resulting in a 5 wt % solution. To this, an equal volume of 2.0 or 2.5 or 3.0 or 4.0 or 5.0 wt % HTCC 3 was added. The hydrogel was allowed to form for 10 min in an incubator set at 37° C. after which the resulting 2.5 wt % PDA, 0.5 or 1.0 or 1.25 or 1.5 or 2.0 or 2.5 wt % HTCC 3 hydrogel was obtained. Thus, four different material compositions were examined, with the PDA component held constant at 2.5 wt %, while the concentration of HTCC 3 was varied from 1.0, 2.0, 3.0, 4.0 and 5.0 wt %. In the case of antibiotic loaded hydrogel, vancomycin was added to the PDA solution to have antibiotic concentration of 6 mg/mL. While preparing the antibiotic loaded hydrogel, the similar procedure was followed where PDA solution containing antibiotic was added to the antibacterial component of the hydrogel HTCC 3. The hydrogels also were prepared by mixing the two polymer solutions taken in a dual barrel syringe and thereby injecting them together.

TABLE 6
Physical properties and gelation time of the hydrogels

| Sample | Wt % PDA | Wt % HTCC 3 | RNH₂/RCHO | t_{Gel} (s) |
|---|---|---|---|---|
| Gel 1 | 2.5 | 1.0 | 0.59 | >30 |
| Gel 2 | 2.5 | 1.5 | 0.87 | 20 |
| Gel 3 | 2.5 | 1.75 | 1.01 | 10 |
| Gel 4 | 2.5 | 2.0 | 1.17 | 5 |
| Gel 5 | 2.5 | 2.5 | 1.46 | <5 |

PDA = Polydextran aldehyde;
t_{Gel} = gelation time;
RNH₂ = (—NH— + —NH₂) of HTCC polymer;
RCHO = bisaldehyde functionality in PDA

Five different hydrogels (Gel 1, Gel 2, Gel 3, Gel 4 and Gel 5) were prepared by varying the concentrations of HTCC (Table 6). The RNH₂/RCHO ratio serves as a convenient parameter to rationalize material performance and is defined by the starting concentrations of PDA and HTCC 3, respectively, before the components are mixed. Table 6 shows that as the RNH2/RCHO ratio increases, the adhesive forms more quickly on mixing of the two components. This is due to the greater number of primary amines available for reaction at higher ratios. The gelation time for the hydrogel was found to be less than 5 seconds in Gel 5 thus indicating that fast hydrogelation of the both PDA and HTCC 3.

### Example 10

### In-Vitro Antibacterial Activity of the Hydrogel:

Hydrogels used for antibacterial assessment were prepared by adding an equal volume (75 μL) of HTCC 3 to 75 μL of a 5 wt % PDA solution with or without antibiotic. A volume of 100 μL of this mixture was immediately transferred to the wells of a 96-well plate. The hydrogels were incubated at 37° C. for 30 min after which all hydrogels were washed to remove any un-cross-linked HTCC 3. First, the hydrogels were rinsed with PBS and then the gel surfaces were washed with nutrient media. For the determination of antibacterial activity of hydrogel with or without the antibiotics, 6 h grown bacterial solution (˜10⁹ CFU/mL) was diluted to 10⁵ CFU/mL in nutrient media and 100 μL was introduced to the surface of the hydrogel surface or tissue culture treated plate (TCTP) as a control. The bacteria were incubated on the hydrogel or TCTP surfaces for 24 h at 37° C. after which OD of the solution above the gel was measured at 600 nm. To calculate the antibacterial activity, OD value of the hydrogel containing only media was also recorded and subtracted from the OD values of the treated samples.

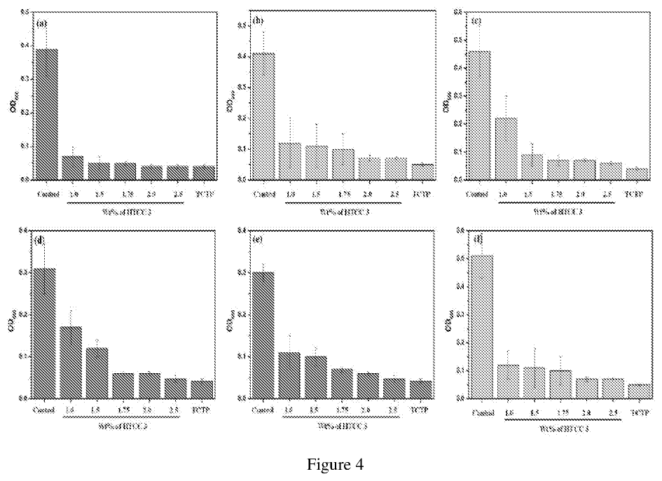

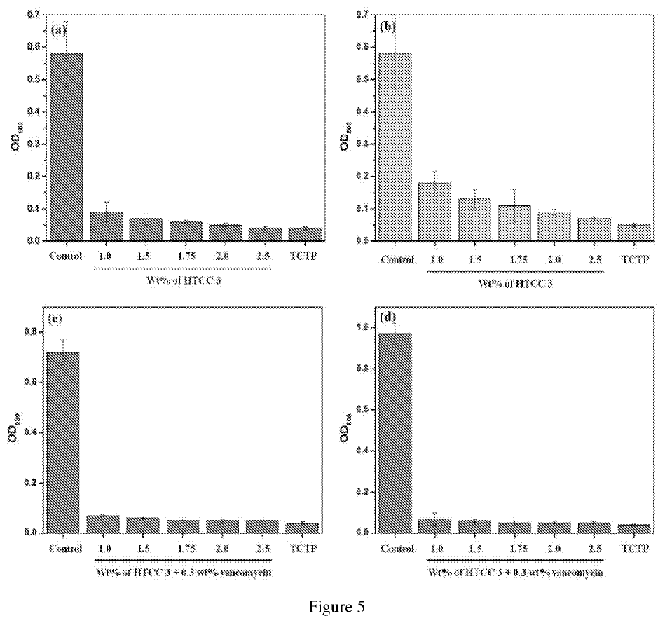

All the hydrogels showed antibacterial activity against both Gram-positive and Gram-negative bacteria. As the weight percent of HTCC 3 increases in the formulation, the antibacterial activity of the hydrogel's surface increases. For example, at 1.0 wt % HTCC 3 the surfaces are moderately active against both Gram-positive and Gram-negative bacteria. However, if the weight percent is increased to 2.5 wt % the surfaces become extremely active (as the OD values of the treated bacterial solution were similar to the OD values of only media thus indicated no growth of the bacteria in the treated sample) (FIG. 4). This HTCC 3 concentration-dependent behavior suggests that the quaternary ammonium group content of the hydrogel directly influences its antibacterial activity. The activity of the adhesive was further studied by challenging its surface with an increasing number of colony forming units (CFUs) of bacteria. It was observed that this hydrogel's surface is extremely active against both S. aureus and E. coli even at 10⁷ CFU/mL and 10⁸ CFU/mL (FIGS. 5a and 5b). This number of bacteria is about million times greater than that of a contaminated operating theatre. In contrast, when the same number of bacteria were introduced to the TCTP control surface, uninhibited growth was observed as the OD values increased (FIGS. 5a and 5b). With the antibiotic loaded hydrogel, all the formulations were found to be extremely active (FIGS. 5c and 5d). Hydrogel with vancomycin (0.3 wt % of vancomycin) showed complete activity with all the formulations against methicillin-resistant S. aureus (MRSA) with an initial count of 10⁸ and 10⁹ CFU/mL thus indicating high synergistic activity of the gel (FIGS. 5c and 5d).

### Example 11

### Contact Active and Release Based Activity of the Hydrogel:

In order to assess that the hydrogels without antibiotics act only by contact based mechanism (i.e., the antibacterial component of the gel, HTCC 3, does not leach out from the gel and kill bacteria only on contact with gel) whereas the hydrogels loaded with antibiotics act by releasing antibiotics in surrounding medium in addition to their contact based activity, the following experiments were performed. Hydrogels were prepared in trans-well cell culture inserts of a 24-well plate at a volume of 350 μL. The hydrogel surfaces were washed by the addition of 700 μL of PBS to the bottom of each well in a 24-well plate, and an additional 100 μL of PBS was added to the top surface. A 10⁹ CFU/mL bacteria stock was prepared as mentioned previously, and diluted to 10⁴ CFU/mL in nutrient media. A volume of 500 μL of this solution was introduced to a given well and the freshly washed hydrogel contained in the trans-well insert was positioned above the bacterial suspension. An additional 100 μL of bacteria-free nutrient media was supplemented to the top of the hydrogel to prevent evaporation. As a control, soluble HTCC 3 and vancomycin at the same concentration and volume was added to a trans-well inserts and incubated above the bacteria. In addition, untreated bacteria were included as a negative control. Sample plates were incubated at 37° C. for a total of 24 h, after which bacterial growth was assessed by measuring the OD values of the solution.

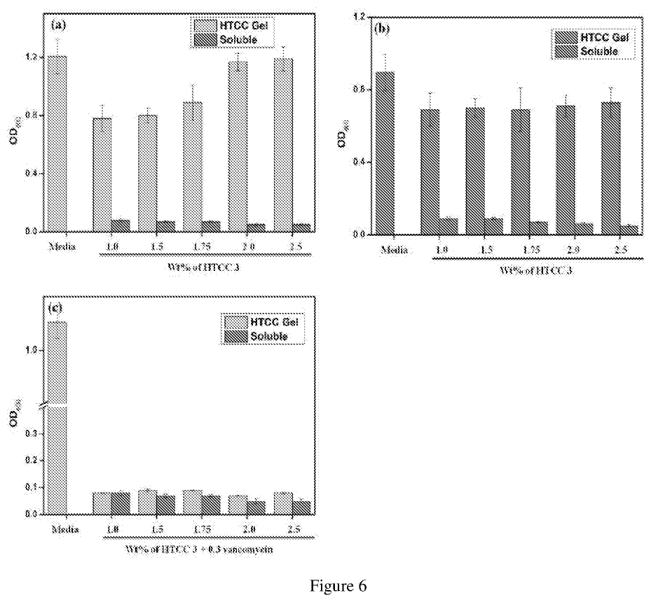

If HTCC 3 is capable of leaching from the adhesive into the culture media, bacterial death should result in the above experiment which will lead to no rise in OD values. After 24 h of incubation, no cell death was observed for both S. aureus and E. coli cultures, indicating that the HTCC 3 had not leached from the adhesive (the OD values for all the hydrogel formulations increased thereby indicating the bacterial growth). In contrast, when soluble HTCC 3 alone was added to the insert, bacterial proliferation was greatly inhibited (the OD values were found to be similar to that of only media) (FIGS. 6a and 6b). Taken together, the data indicate that the bioadhesive, is itself, inherently antibacterial and act on contact based mechanism. However, in case of antibiotic loaded hydrogel, both hydrogel and the polymer in solution showed complete inhibition of bacterial growth against MRSA thereby indicating that the vancomycin loaded hydrogel released the antibiotic into the solution over time and killed the bacteria in solution (FIG. 6c). Thus, the antibiotic loaded hydrogel act by both release-active and contact-active mechanism.

### Example 12

### Hemolytic Activity of the Hydrogel:

Hydrogels were prepared and rinsed, with PBS only, as indicated previously. Human red blood cells (hRBCs) were isolated from blood samples donated by healthy volunteers. The hRBCs were separated from the plasma and washed three times with sterile PBS by centrifugation at 3500 rpm for 5 min. Next, hRBCs were suspended in PBS resulting in a 5% (v/v) cell suspension. One hundred microlitres of hRBCs were added to the surface of the hydrogels or a control TCTP surface. As a positive control, hRBC suspension was incubated with 0.1% Triton-X. The plate was incubated at 37° C. for 1 h and then the plate was centrifuged at 3500 rpm for 5 min after addition of hundred microlitres of PBS. The supernatant (100 μL) was transferred to another 96-well plate and then OD value of the supernatant was recorded at 540 nm. Hemolytic activity was assessed by measuring the amount of haemoglobin liberated to the surrounding solution due to membrane rupture. Controls defining 0 and 100% haemolysis were hRBCs plated in PBS on TCTP in the absence or presence of 0.1% Triton-X, respectively. For phase-contrast imaging, after 1 h incubation, the suspension above the gels and TCTP were mixed gently by pipetting 10 μL of the suspension was transferred to wells of a 96-well plate containing 90 μL of PBS. Images were collected on a Leica DM IL LED microscope.

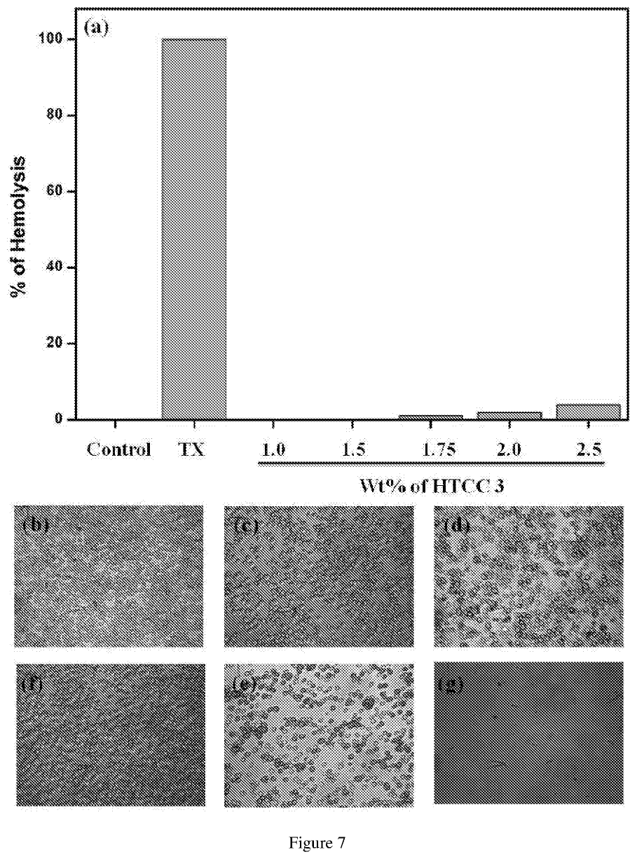

The hydrogels were found to be completely non-hemolytic. The potential of the hydrogel to lyse red blood cells increases negligibly with HTCC content. For adhesives composed of 2.5 wt % HTCC or less, the percent hemolysis is within the guidelines established by the ASTM (ASTM-F756) (only 2-4% hemolysis was observed) (FIG. 7a). When the treated RBCs were imaged, erythrocytes resting on the TCTP control surface as well as the different adhesive surfaces display typical healthy round morphology, supporting the assertion that these surfaces are non-haemolytic. In contrast, when the detergent Triton-X is added, full hemolysis is evident (FIG. 7b).

### Example 13

### In-Vivo Activity of the Hydrogel (Cecal Ligation and Puncture Model of Sepsis Prevention in Mice):

In order to assess the utility of the hydrogel's biological activity, e.g, to prevent in-vivo sepsis and to show its adhesive properties, mice sepsis model of cecal ligation and puncture experiment was performed with the hydrogels. Six- to eight-week-old male C57BL/6 mice were used for this experiment (n=8 per group). Animals were anaesthetized with ketamine-xylazine and the abdomen was then shaved and disinfected by first applying betadine solution followed by wiping with a 70% alcohol swab. All procedures were performed under clean but not sterile conditions. A 1.5 cm midline laparotomy was performed to expose the cecum. Next, the cecal pole was tightly ligated with a 6.0 silk suture at 0.5 cm from its tip, and then perforated once with a 20-gauge needle. In the experimental group, the cecum was covered with the adhesive gel (2.5 wt % PDA cross-linked with 2.5 wt % HTCC 3 or 2.5 wt % PDA containing 0.6 wt % of vancomycin cross-linked with 2.5 wt % HTCC 3) before returning it back to the peritoneal cavity, whereas in the control group the cecum was directly returned to the peritoneal cavity. The abdominal wall was then closed in layers using a 6.0 silk running suture for the peritoneum and a 6.0 nylon suture for the skin. All animals were resuscitated by injecting 1 mL of 0.9% saline solution subcutaneously. Buprenorphine (0.05 mg/kg) was injected subcutaneously for postoperative analgesia. The animals were then placed on a heating pad until full recovery. Free access to food and water was ensured post-surgery. Mice were monitored every 12 h for survival and weight loss.

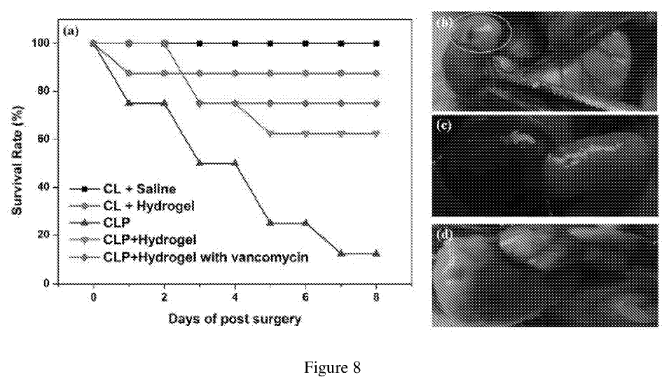

Application of the adhesive gel to the puncture area should form a barrier between the cecum and peritoneal cavity, and inhibit bacterial infiltration/translocation. FIG. 8a contains survival curves that showed that five out of eight mice treated with adhesive survived (63%) until the termination of the study at day 8. Only one mouse in the control group survived (13%) over this same time. The FIG. 8 also shows that when gel is administered to animals without punctures, survival is high. It was also observed that six out of eight mice treated with adhesive survived (75%) until the termination of the study at day 8. FIG. 8b showed that when the adhesive is applied to the puncture area, a thin film forms over the resulting haematoma that seals the cecum. FIGS. 8c and 8d shows representative gross anatomical pictures of control and experimental cecum isolated from animals 24 h after the start of the experiment. The control cecum to which a puncture was made but no adhesive administered was highly erythematous and appeared dark in colour, indicating severe gross infection. In contrast, the experimental punctured cecumto which adhesive had been applied appeared healthy and normal in colour, indicating that the gel had formed an effective barrier to infection.

### Example 14

### In-Vivo Hemostatic Ability:

To evaluate the hemostatic potential of the hydrogels, a hemorrhaging liver mouse model was employed (C57BL/6 mouse, 22-25 g, 6-8 weeks, male). A mouse was anesthetized using ketamine-xylazine mixture and fixed on a surgical corkboard. The liver of the mouse was exposed by abdominal incision, and serous fluid around the liver was carefully removed to prevent inaccuracies in the estimation of the blood weight obtained by the filter paper. A pre-weighted filter paper on a paraffin film was placed beneath the liver. Bleeding from the liver was induced using a 20 G needle with the corkboard tilted at about 30° C. and 50 μL of the hydrogel was immediately applied to the bleeding site using the dual barrel syringe filled with the HTCC 3 and PDA solutions (50 mg/mL each). After 3 min, the weight of the filter paper with absorbed blood was measured and compared with a control group (no treatment after pricking the liver).

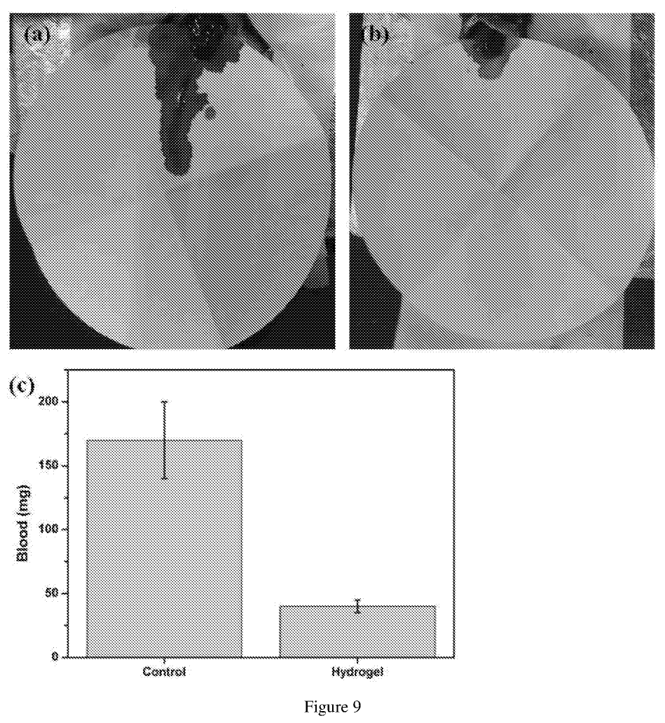

FIGS. 9a and 9b show photographs of untreated bleeding liver and the extent of bleeding after the application of hydrogels onto the liver, respectively. The total blood loss from the control liver was about 175 mg for 3 min after the liver was pricked with a needle. In contrast, the bleeding was significantly arrested by the dressing of hydrogels, the loss of blood being reduced to 35 mg through the combined effect of the adhesiveness and the hemostatic property of the hydrogels (FIG. 9c). This result demonstrates that the hydrogels exhibit both elastic and adhesive properties when crosslinked in situ, thus serving as an effective anti-hemorrhaging agent.

### Example 15

### Synthesis of Water-Soluble Quaternary Chitosan Derivatives:

Synthesis of Phthaloylated Chitosan:

Chitosan (degree of acetylation=85%) (5 g) was taken in a round bottomed glass. To the polymer, phthalic anhydride (15.3 g) was added and anhydrous N,N-dimethyl formamide (DMF, 100 mL) was added to the mixture. The mixture was then purged with argon and heated at 130° C. for about 8 h with constant stirring under the argon atmosphere. After the reaction, the reaction mixture was poured into ice-cold water to precipitate the phthalimide protected chitosan. The precipitate was filtered off with sintered glass funnel and washed with methanol to remove unreacted phthalic anhydride. The product was dried in vacuum oven at 55° C. for about 24 h.

All the free amine groups of chitosan were reacted with phthalic anhydride and the presence of phthalic anhydride moiety was confirmed and quantified by FT-IR and ¹H-NMR spectroscopy. The IR spectra revealed the presence of benzene group at 1590 cm⁻¹. NMR spectra revealed the presence of aromatic moiety at 7.789 ppm and the degree of phthylation was found to be ˜84±1%.

Synthesis of Phthaloylated Tosyl Chitosan:

Phthaloylated chitosan (2.0 g) and lithium chloride (LiCl, 5.2 g) dried at 80° C. overnight and at 130° C. for 4 h respectively and then were taken in a two-necked round bottom flask fitted with rubber septa. The flask was purged with oxygen-free nitrogen, and anhydrous N,N-dimethylacetamide (DMAc) (104 mL) was added. The mixture was then stirred at room temperature until all the solids were dissolved. Dry NEt₃ (20 mL) was added to the the flask was transferred to a cold reaction chamber at 8° C. A solution of tosyl chloride (27 g) in DMAc (48 mL) was added to the reaction mixture and the reaction was allowed to proceed for 48 h at the same temperature. The insoluble solid from the reaction mixture was filtered and to the filtrate, excess ice-cold water was added to obtain phthaloylated tosyl chitosan. The precipitate was filtered and washed successively with water, ethanol and ether to obtain the product.

Presence of tosyl group was confirmed and quantified by FT-IR and ¹H-NMR spectroscopy. The IR spectra revealed the presence of the tosyl group at 1710 cm⁻¹ (SO₂, symmetric) and NMR spectra revealed the presence of aromatic moiety of tosyl group at 7.2 ppm and 7.6 ppm. The degree of the tosylation (DS) was calculated as the ratio of sulfur by nitrogen obtained in elemental analysis (DS=S/N×100%) and was found to be ˜79±2%

Synthesis of N,N-dimethylhexylamine Quaternized Chitosan Tosylate:

Phthaloylatedtosyl chitosan (1.0 g) was dissolved in anhydrous N,N-dimethyl acetamide (DMAc) (30 mL) in sealed screw-top pressure tube. To the reaction mixture N,N-dimethylhexylamine (10 equivalent per tosylated sugar unit) was added and the reaction was allowed to proceed at 120° C. for 96 h. After the reaction, diethyl ether was added in excess (150 mL) to precipitate the quaternized chitosan derivatives. The precipitate was filtered through a sintered glass funnel and was washed repeatedly with diethyl ether to obtain pure quaternary derivative with 100% degree of quaternization (with respect to tosyl groups for each tosylchitosan).

Presence of tosylate anion was confirmed by FT-IR spectroscopy. The IR spectra revealed the presence of the tosylate group at 1380 cm⁻¹ (SO₂, asymmetric) and 1710 cm⁻¹ (SO₂, symmetric). Complete quaternization was confirmed from ¹H-NMR as the spectra revealed only two peaks at 7.041 ppm and 7.501 ppm corresponding to tosylate anion.

Synthesis of N,N-dimethylhexyl Ammonium Chitosan Tosylate:

Quaternized chitosan (0.3 g) was suspended over 15 mL 50 wt % hydrazine solution and stirred at 100° C. for 18 h under Ar atmosphere. After that the hydrazine solution was evaporated and the rest of the reaction mixture was dissolved in methanol. In the methanol solution acetone was added to precipitate the final chitosan derivative and the precipitate was washed with acetone repeatedly to get the N,N dimethyl ammonium chitosan tosylate.

Deprotection of the phthalimide group was confirmed from the ¹H-NMR spectroscopy. Absence of the peak at 7.78 ppm reveals the complete deprotection of the protected amino group in the chitosan derivative. ¹H-NMR: (400 MHz, D₂O, δ): 0.83 (bs, —CH₃(CH₂)₅—N⁺(CH₃)₂—, 3H), 1.25 (m, —CH₃(CH₂)₃CH₂CH₂—N⁺(CH₃)₂—, 6H), 1.69 (m, —CH₃(CH₂)₃CH₂CH₂—N⁺(CH₃)₂—, 2H), 2.03 (s, —NHCOCH₃), 2.33 (s, SO₃—C₆H₄—CH₃), 2.83-3.89 (m, Cell-H and —CH₃(CH₂)₃CH₂CH₂—N⁺(CH₃)₂—), 7.30 (d, SO₃—C₆H₄—CH₃, m-H), 7.64 (d, SO₃—C₆H₄—CH₃, o-H).

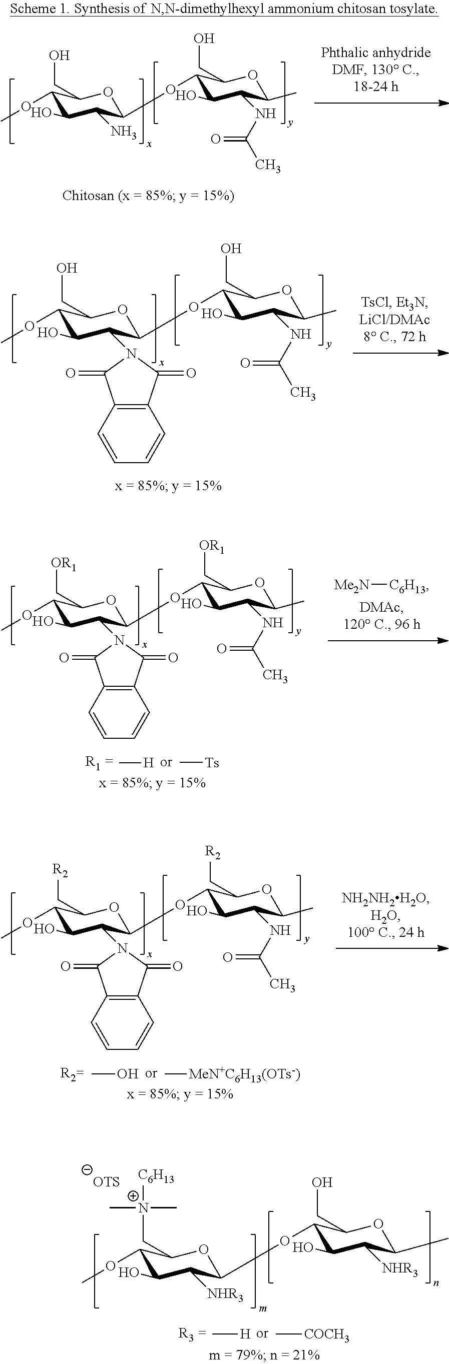

Synthesis of N,N-dimethyloctylamine Quaternized Chitosan Tosylate:

Phthaloylatedtosyl chitosan (1.0 g) was dissolved in anhydrous N,N-dimethyl acetamide (DMAc) (30 mL) in sealed screw-top pressure tube. To the reaction mixture N,N-dimethylhexylamine (10 equivalent per tosylated sugar unit) was added and the reaction was allowed to proceed at 120° C. for 96 h. After the reaction, diethyl ether was added in excess (150 mL) to precipitate the quaternized chitosan derivatives. The precipitate was filtered through a sintered glass funnel and was washed repeatedly with diethyl ether to obtain pure quaternary derivative with 100% degree of quaternization (with respect to tosyl groups for each tosylchitosan).

Presence of tosylate anion was confirmed by FT-IR spectroscopy. The IR spectra revealed the presence of the tosylate group at 1380 cm⁻¹ (SO₂, asymmetric) and 1710 cm⁻¹ (SO₂, symmetric). Complete quaternization was confirmed from ¹H-NMR as the spectra revealed only two peaks at 7.041 ppm and 7.501 ppm corresponding to tosylate anion.

Synthesis of N,N-dimethyloctyl Ammonium Chitosan Tosylate:

Quaternized chitosan (0.3 g) was suspended over 15 mL 50 wt % hydrazine solution and stirred at 100° C. for 18 h under argon atmosphere. After that the hydrazine solution was evaporated and the rest of the reaction mixture was dissolved in methanol. In the methanol solution acetone was added to precipitate the final chitosan derivative and the precipitate was washed with acetone repeatedly to get the N,N-dimethyl ammonium chitosan tosylate.

Deprotection of the phthalimide group was confirmed from the ¹H-NMR spectroscopy. Absence of the peak at 7.78 ppm reveals the complete deprotection of the protected amino group in the chitosan derivative. ¹H NMR: (400 MHz, D₂O, δ): 0.872 (bs, —CH₃(CH₂)₇—N⁺(CH₃)₂—, 3H), 1.27 (m, —CH₃(CH₂)₅CH₂CH₂—N⁺(CH₃)₂—, 10H), 1.73 (m, —CH₃(CH₂)₅CH₂CH₂—N⁺(CH₃)₂—, 2H), 2.07 (s, —NHCOCH₃), 2.32 (s, SO₃—C₆H₄—CH₃), 2.94-3.93 (m, Cell-H and —CH₃(CH₂)₅CH₂CH₂—N⁺(CH₃)₂—), 7.27 (d, SO₃—C₆H₄—CH₃, m-H), 7.68 (d, SO₃—C₆H₄—CH₃, o-H).

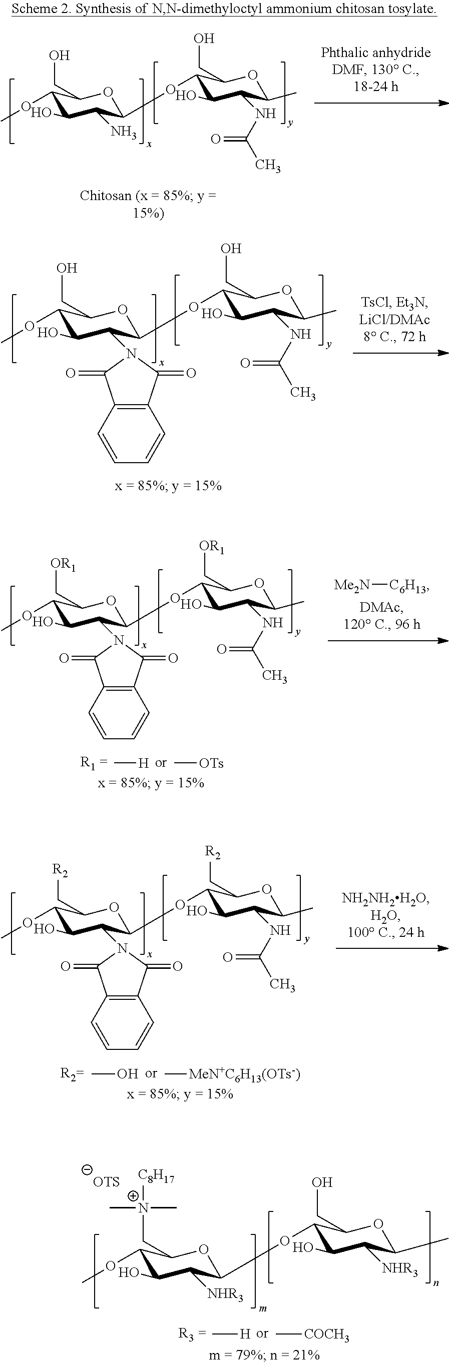

### Example 16

### Wound Healing Activity of the Hydrogel:

The wound healing abilities of the injectable hydrogels were performed in a rat model. Studies with the rats were performed according to protocols approved by the Institutional Animal Ethics Committee (IAEC) in the institute (Jawaharlal Nehru Centre for Advanced Scientific Research). Wistar rats (male, 250-300 g) were used for the experiment. Animals were divided into two groups: control and test groups. In each group 5 rats were used. The animals were anesthetized by intraperitoneal injection of the cocktail of ketamine (40-50 mg/kg) and xylaxin (2-3 mg/kg) body weight. Skin above the dorsal midline of the animals was shaved aseptically. Wounds of 18 mm diameter were prepared by excising the dorsum of the rats. The hydrogel (containing 2.5 wt % PDA and 2.5 wt % HTCC, 400 μL) was then applied at the wound site via a syringe after immediate mixing of both the components. Then gels were spread on the entire wound area with the help of a glass rod. The rats of the tests groups were covered with sterile gauze. Then elastic adhesive bandage (Dynaplast, Johnson & Johnson) was used to fix the gauze. Wounds were also covered with the gauze and fixed with adhesive bandage without gel and used as controls. The animals were then kept in separate cages and allowed to have access of food and water. After the predetermined time interval (after postsurgical day 5, 10, 15 and 20) rats were sacrificed. Finally, wounds were grossly observed and photographed to measure the reduction of wound size.

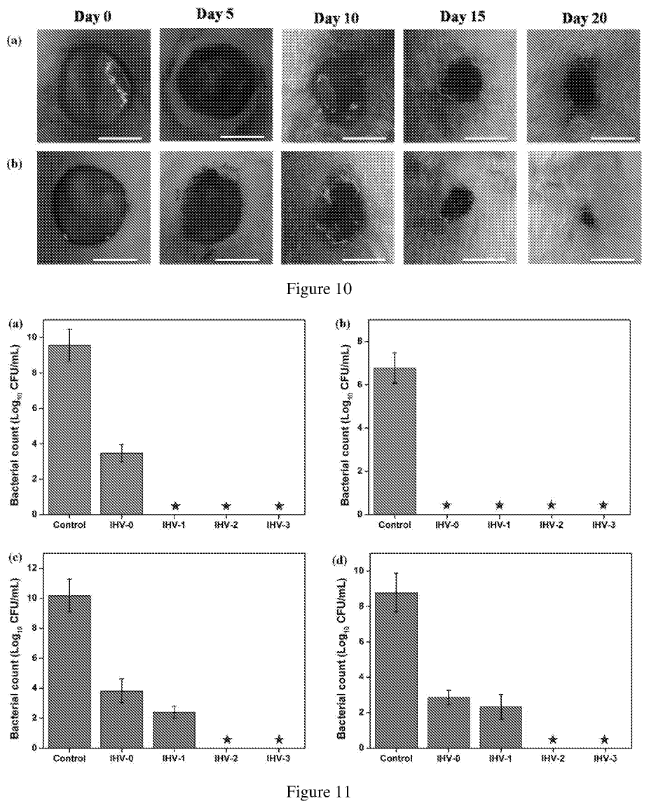

While the control group have no significant effects on the wound size after day 5 and day 10, gel with 2.5 wt % HTCC and 2.5 wt % PDA was able to reduce the wound size by 14±4% and 31±8%, respectively [FIGS. 10 (a) and 10(b)]. Notably, after day 15 and day 20, wound in the treated groups almost repaired in sharp contrast to control. The adhesive has a wound healing ratio of 78±9%, much higher than that of the control group (58±7%) (FIGS. 10a and b). In summary, the adhesive exhibited excellent wound-healing performance over a very short period and may find potential applications in clinical settings. The scale bar was 10 mm as represented in FIG. 10.

### Synthesis of Antibiotic-Loaded Hydrogels:

Vancomycin was dissolved in phosphate buffer (23.5 mM NaH₂PO₄, 80.5 mM Na₂HPO₄) at different amounts (1 mg/mL, 6 mg/mL and 12 mg/mL). To this PDA was added to obtain PDA solution (50 mg/mL) containing vancomycin in the above mentioned concentration (5 wt % PDA, 0.1 wt %, 0.6 wt % and 1.2 wt % vancomycin respectively). After 1 h, an equal volume of 40 mg/mL HTCC (4.0 wt %) dissolved in Millipore water was added to the vancomycin-containing PDA solution. The mixture was then kept in an incubator for 15 min at 37° C. to allow gel formation. This resulted in various gel formulations (IHV-1: 2.5 wt % PDA with 0.05 wt % vancomycin and 2.0 wt % HTCC; IHV-2: 2.5 wt % PDA with 0.3 wt % vancomycin and 2.0 wt % HTCC; IHV-3: 2.5 wt % PDA with 0.6 wt % vancomycin and 2.0 wt % HTCC). Hydrogel without any vancomycin (IHV-0) was prepared by adding 5 wt % PDA solution to 4 wt % HTCC solution. The gels were prepared directly in the wells of a 96-well plate or in a sample vial or in petri dish either by simple mixing of the solutions or by mixing via dual barrel syringe.

The extent of covalent conjugation of vancomycin to PDA and hence in the gel was determined as follows: PDA and vancomycin mixture (PDA at 50 mg/mL and vancomycin at 1 mg/mL or 6 mg/mL or 12 mg/mL) in phosphate buffer (5 mL) and kept at 37° C. for 1 h in the dark. The solution was then dialyzed for 6-8 h using dialysis membranes (molecular cut off 3500 kDa) at 4° C. in deionized water in the dark by changing water on a regular interval with 30 min between each water change. The solution was then freeze dried and stored at 4° C. under dark condition. ¹H-NMR and FT-IR spectra of the freeze dried sample were then recorded in deuterated solvent and KBr pellets respectively. The freeze dried samples were also used for elemental analyses after drying in a vacuum over at 55° C.

TABLE 7
Physical properties of the various hydrogel formulations

| | Wt % | Wt % | Wt % | t_{gel} | G′ |
|---|---|---|---|---|---|
| Hydrogel | PDA | HTCC | Vancomycin | (s) | (Pa) |
| IHV-0 | 2.5 | 2.0 | 0 | 10-12 | 830 ± 120 |
| IHV-1 | 2.5 | 2.0 | 0.05 | 10-15 | 847 ± 87 |
| IHV-2 | 2.5 | 2.0 | 0.3 | 10-15 | 887 ± 141 |
| IHV-3 | 2.5 | 2.0 | 0.6 | 10-15 | 849 ± 110 |

### Example 18

### In-Vitro Antibacterial Activity of Antibiotic-Loaded Hydrogels:

First the hydrogels with or without antibiotic were prepared in the wells of a 96-well plate (50 μL 50 mg/mL of PDA containing 1 mg/mL or 6 mg/mL or 12 mg/mL of vancomycin and 50 μL 40 mg/mL of HTCC). The plate was then kept for 10-15 min in an incubator to allow the gel formation. To the wells bacteria (150 μL of ˜10⁵ CFU/mL or 10⁷ CFU/mL of S. aureus and MRSA) were added. The plates containing bacteria were then incubated at 37° C. for about 6 h under constant shaking. After incubation, 50 μL of bacterial suspension was either directly plated or diluted following 10-fold serial dilution and then plated on suitable agar plate. The agar plates were then incubated for about 24 h at 37° C. Finally, bacterial colonies were counted to evaluate the reduction in viable cells. The presence of viable bacterial count in each case was then expressed as log CFU/mL. A similar experiment was performed with the blank wells without any gels and hydrogel without any vancomycin as controls.

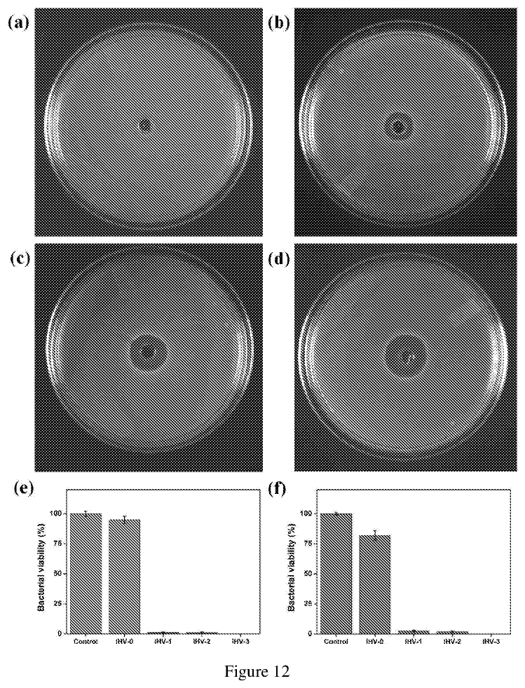

While the blank wells showed 9.5±0.9 log CFU/mL S. aureus, IHV-0 showed 3.4±0.5 log CFU/mL bacteria after 6 h of incubation. Interestingly, gels with vancomycin (IHV-1, IHV-2 and IHV-3) showed no survival of bacteria thus demonstrating the superior efficacy in killing S. aureus (FIG. 11a). Similar results were obtained for the drug-resistant bacteria when the gels were challenged with MRSA (150 μL, 1.2×10⁴ CFU/mL). While the blank wells were shown to have 6.8±0.7 log CFU/mL MRSA after 6 h, IHV-1, IHV-2 and IHV-3 showed complete killing of MRSA (FIG. 11b). Efficacy of the vancomycin-containing hydrogels was further established by challenging the gel's surface with higher amount of bacteria. Interestingly while the blank wells showed 10.2±1.1 log CFU/mL of bacteria after 6 h, gels with no or less antibiotic such as IHV-0 and IHV-1 showed 3.8±0.8 log CFU/mL and 2.4±0.4 log CFU/mL of bacteria when incubated with an initial amount of 1.67×10⁷ CFU/mL of S. aureus. However, no bacteria were observed for the gels with higher amount of antibiotic, e.g., IHV-2 and IHV-3 (FIG. 11c). Not only against drug-sensitive bacteria, the antibiotic-loaded gels showed remarkably higher activity than gels with no antibiotic. For example, for an initial amount of 1.1×10⁶ CFU/mL of MRSA, while the blank wells showed 8.7±1.1 log CFU/mL of MRSA after 6 h, IHV-0 and IHV-1 showed 2.8±0.4 log CFU/mL and 2.3±0.7 log CFU/mL of bacteria. IHV-2 and IHV-3, on the other hand, showed no bacteria thus indicating complete eradication of MRSA (FIG. 11d). Stars in FIG. 11 represent less than 50 CFU/mL.

### Example 19: Diffusion of Antibiotic from the Hydrogel (Zone of Inhibition)

Nutrient agar gels were prepared in petri dishes (90 mm) according to the manufacturer's protocols. Briefly, 2.5 g of nutrient agar was dissolved in 100 mL of Millipore water and then autoclaved for 15-18 min at 121° C. After cooling to 50° C., a volume of 12-15 mL of the agar solution was added to the petri dishes and allowed to cool to room temperature, resulting in solid agar gel. A circular piece (6 mm in diameter) of the agar gel was removed by incision to reveal the underlying polystyrene. A 50 μL of IHV-0, IHV-1, IHV-2 and IHV-3 gel was then prepared in the cavity of agar plates following the method as mentioned previously. The hydrogel was incubated at 37° C. for 15 min, after which the gel surfaces were washed three times with 5 mL of PBS to remove any non-cross-linked HTCC and to ensure that the pH was equilibrated. A volume of 1 mL of 10⁸ CFU/mL of S. aureus and MRSA was added to each dish and gently rocked to provide the full surface coverage. The plates were then incubated for 24 h imaged by Cell biosciences gel documentation instrument.

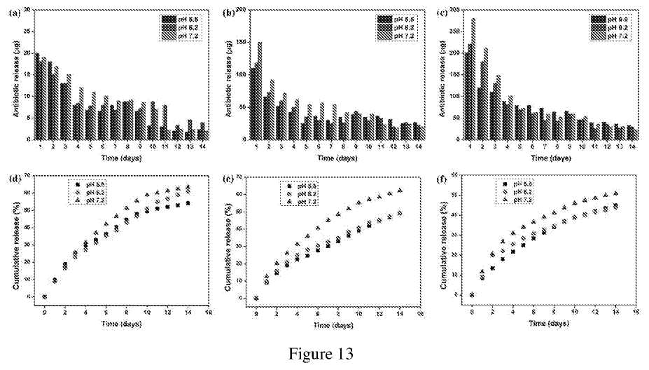

As expected IHV-0 did not show any zone of clearance though it showed no colonies on the gel's surface thus inactivate bacteria only upon contact (FIG. 12a). IHV-1, IHV-2 and IHV-3, on the other hand, displayed significant zone of inhibition against MRSA lawns grown on the agar plate thereby indicating the diffusion of vancomycin to the surroundings which inactivated bacteria in the respective areas (FIG. 12b-d). Furthermore, IHV-3 with highest amount of encapsulated antibiotic showed maximum zone of inhibition while IHV-1 with lowest amount of encapsulated drug showed minimum inhibition zone.

### Example 20

Released Based Activity from Antibiotic-Loaded Hydrogels:

Hydrogels (400 μL) were prepared in the inserts of a trans-well cell culture plate (24-well). The surfaces of the gels were washed by PBS (1 mL) to the bottom of the wells in the 24-well plate. PBS (100 μL) was added onto the surface of the gel. The plates were then kept for 15 min in an incubator set at 37° C. and the PBS solutions from the bottom and top of the gels were removed. Similarly, the gels were further washed two more times. Bacteria (500 μL, ˜10⁴ CFU/mL of S. aureus and MRSA) were added to the bottom of the wells of trans-well cell culture plate and then the inserts containing the hydrogels were placed above the bacterial suspension. Nutrient media without bacteria (100 μL) was also added onto the surface of the hydrogel. A control was made where only bacteria (500 μL, ˜10⁴ CFU/mL of S. aureus and MRSA) were incubated. Then the well plate was incubated at 37° C. for about 24 h. Finally, bacterial growth was determined by measuring the OD values of the bacterial suspension. Cell viability was then calculated with respect to the OD values of the control wells and taking it as 100% bacterial growth.

Like the control wells, substantial bacterial growth was observed in the wells with inserts containing IHV-0 after 24 h. If there was any release of HTCC from IHV-0, the bacterial growth should have been inhibited. The above results thus suggested that IHV-0 is capable of reducing bacterial count only on contact [FIG. 12(a)-12(f)]. Interestingly, wells with inserts that contained IHV-1, IHV-2 and IHV-3 showed no growth of bacteria thus indicated that these gels released vancomycin in the bottom solution leading to bacterial inhibition (FIG. 12(a)-12(f)).

### Example 21

### Long Lasting Antibacterial Activity of Antibiotic-Loaded Hydrogels:

Hydrogel (IHV-2) was prepared in of eppendorf tube (2 mL) by mixing the components (200 μL of 50 mg/mL PDA with 6 mg/mL vancomycin and 200 μL of 40 mg/mL HTCC). After the preparation, 1 mL of PBS buffer or nutrient media was added on top of the gel. Then the gel with the added liquids was kept for constant shaking at 37° C. for 24 h. After 24 h, the buffer or media was collected and replaced with the fresh buffer. The process was repeated for next 14 days. Finally, the antibacterial activity of the released vancomycin was determined by taking 450 μL of the buffer or media with 50 μL of ˜10⁷ CFU/mL MRSA. The bacterial mixture was kept for 24 h and then OD value was recorded. Also, the released media-bacterial solution was directly spot plated on agar plate to determine the bactericidal effect of the released vancomycin.

Notably, the release media inactivated MRSA completely till 14 days as tested. These results thus portrayed the utilities of the vancomycin-loaded hydrogels as dual action drug-carrier with effective and sustained release properties.

### Example 22

In-Vitro Release Kinetics of Vancomycin from Antibiotic-Loaded Hydrogels:

Hydrogel (IHV-2) was prepared in eppendorf tube (2 mL) in a similar way as described previously (200 μL of 50 mg/mL PDA with 6 mg/mL vancomycin and 200 μL of 40 mg/mL HTCC). After the preparation, 1 mL of phosphate buffer of varying pH (5.5, 6.2 and 7.2) was added on top of the gel. Then the gel with added buffer was kept for constant shaking at 37° C. for 24 h. After 24 h, the buffer was collected and replaced with the fresh buffer. The process was repeated for 14 days. Finally, the amount of released vancomycin was determined by UV-visible absorption spectroscopy. A standard calibration curve of absorption intensity versus concentration was generated for vancomycin (absorbance at 281 nm). The concentration of the released vancomycin was then determined after measuring the absorbance and fixing the value in the absorption intensity versus concentration curve.

Notably, gels were shown to release the antibiotic continuously over 14 days as tested in all three pH (FIG. 13). Interestingly, release of vancomycin was found to be dependent on the initial amount of loading (FIG. 13a-c). Further, the release kinetics was also found to be dependent on pH of the buffer. At higher (pH 7.2), higher amount of vancomycin was found to be released. For example, while IHV-2 showed ˜49-50% release at pH 5.5 and 6.2, 62% release was observed at pH 7.2 after 14 days (FIG. 6d-f). It should be mentioned that after 11 days, the UV-absorption spectra of the collected buffer solutions showed the presence of a weak absorption peak at 290-300 nm in addition to a peak at 281 nm at lower pH (pH 5.5 or 6.2). This indicated that a minor amount of PDA-van conjugates with imine bonds got released from the gels along with the free antibiotics. Thus, the studies were conducted till 14 days at all three pH's. It should also be mentioned that the IHV-2 and IHV-3 gels released slightly higher proportion of antibiotic in the first 1-2 days possibly by combined release of the covalently bonded vancomycin as well as diffusion of non-covalently bonded antibiotic (FIG. 13). However, the rate of release was found to be almost linear for all three formulations possibly due to the drug release being mostly controlled by the opening of the covalent imine bonds. In general, the above results indicated that an extended release of the drug was achievable by encapsulating antibiotics in the hydrogel network. Interestingly, gels with higher amount of vancomycin (IHV-2 or IHV-3) were shown to release the antibiotic till 40 days (at pH 7.2) which indicated the effectiveness of the matrix in controlling the release behavior of the antibiotic.

### Example 23

### In-Vivo Activity (Subcutaneous Infection of MRSA) of Antibiotic-Loaded Hydrogels:

Female BALB/c mice (6 to 7 weeks old, 18-21 g) were used for the experiment. The mice were first rendered neutropenic (˜100 neutrophils/mL) by injecting cyclophosphamide, i.p. (first dose at 150 mg/kg and then second dose at 100 mg/kg after 3 days of the first dose). Fur above the thoracic midline of each animal was clipped. Then hydrogel (2.5 wt % PDA with 0.3 wt % vancomycin and 2.0 wt % HTCC, 100 μL) was injected subcutaneously. Then MRSA (˜10⁷ CFU/mL, 40 μL) was injected directly into the gel. In another group of mice, bacteria were injected at a distal site (1.5-2.0 cm) from the gel. In another group, bacteria (100 μL saline+40 μL ˜10⁷ CFU/mL MRSA) were injected subcutaneously below the thoracic midline. After 3 days, the animals were sacrificed and surrounding tissues were collected. Tissue samples were then homogenized, and used for cell counting by plating the homogenized solution on nutrient agar plate followed by 10-fold serial dilution. The MRSA count was then expressed as log CFU/g of tissue and expressed as mean±standard error of mean. A small section of the skin tissue from the injection site was also fixed in 10% formalin to study the histological responses.

Once the infection was clinically evident in the control mice (after 72 h), both the control and experimental mice were killed and infection sites were imaged. While significant amount of pus formation was noticed when MRSA was injected in mice (FIG. 13a), no pus formation was observed when bacteria were injected into the gel (FIG. 13b). Interestingly, no pus formation was observed even when the bacteria were injected at a distal site from the gel thereby suggesting that the antibiotic got released from the gel and reduced the viable bacteria (FIG. 13c). To quantify the ability of the vancomycin-loaded gel in eradicating infections, we determined the number of viable bacteria in the mice tissues where hydrogel and/or bacteria were injected. First, the infected tissues were collected and subsequently homogenised. Finally, the tissue lysate was then plated on suitable agar plate and enumerated for bacterial count. When bacteria were injected directly onto the gel IHV-2, tissue surrounding IHV-2 showed 6.1 log less MRSA (99.9999% reduction) as compared to the non-treated tissue sample (while the non-treated tissue showed ˜9.9 log CFU/g of MRSA, the gel treated tissue sample showed 3.8 log CFU/g of MRSA) (FIG. 13d). Most importantly, 5.8 log (99.999%) reduction of MRSA was observed for IHV-2 gel when bacteria were injected at a distal site (the gel and infection site were separated by ˜1.5-2.0 cm) (FIG. 13d). This is possible due to the gradual release of the antibiotic in the surroundings over time thus leading to the inhibition/clearance of bacterial growth even when the bacteria were injected far from the gel.

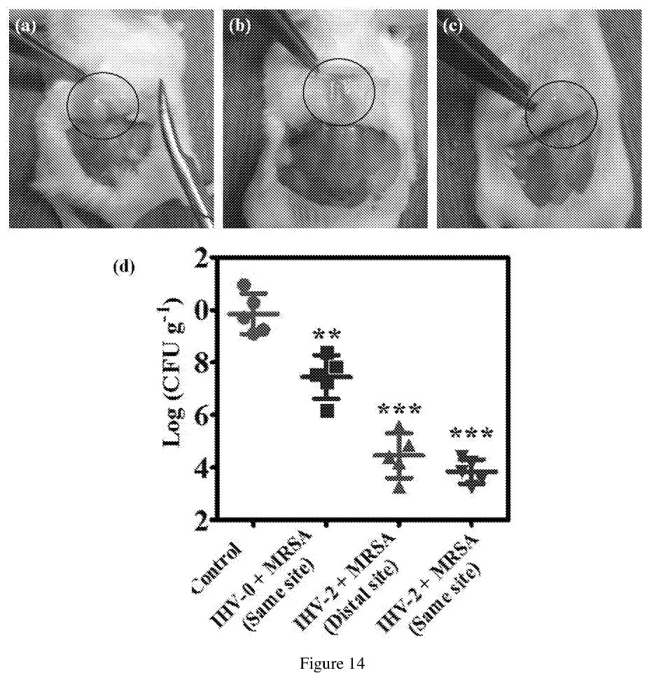

Further, FIG. 14 provides the In-vivo antibacterial efficacy with direct injection of bacteria. Gross internal anatomical images of mice injected subcutaneously with 10⁷ CFU/mL of MRSA (a) directly into the back; (b) into adhesive IHV-0 and (c) into adhesive IHV-2, all after 3 days. Blue circles indicate the site of application. Evaluation of antibacterial activity upon injection of MRSA subcutaneously in mice: (d) MRSA count after 72 h of infection at different conditions; p values (*) are 0.002, <0.0001 and <0.0001 for IHV-0, IHV-2 (same site) and IHV-2 (distal site) samples.

### Advantages

The above mentioned implementation examples as described on this subject matter and its equivalent thereof have many advantages, including those which are described.

1. The disclosed injectable antibacterial hydrogels find use in various biomedical applications such as bio-adhesive materials, local delivery of antibiotics and prevention of infections.
2. The disclosed hydrogel was found to be active against both drug-sensitive and drug-resistant Gram-positive and Gram-negative bacteria.
3. The hydrogel also acts as a sealant and prevents sepsis.
4. The disclosed hydrogels with or without antibiotic were found to be non-toxic towards mammalian cells.
5. The disclosed hydrogels were found to be as effective hemostatic agents
6. The hydrogels were also found to be effective in loading and releasing bioactive molecules, e.g., antibiotics.

Although the subject matter has been described in considerable detail with reference to certain embodiments thereof, other embodiments are possible. As such, the spirit and scope of the invention should not be limited to the description of the embodiment contained herein.

I/We claim:

1. A polymer network comprising a compound of 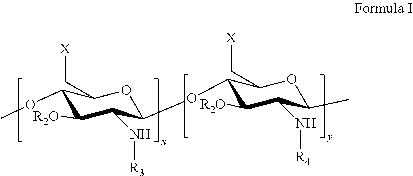
cross-linked to a compound selected from the group consisting of a compound of Formula II; 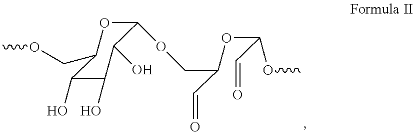
hyaluronate aldehyde, alginate aldehyde, dextran aldehyde, starch aldehyde, and chitosan aldehyde
wherein,
X is selected from the group consisting of OR₁, and 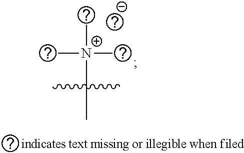
R₁ is selected from the group consisting of hydrogen, and 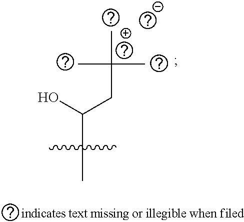
R₂ and R₄ are independently selected from the group consisting of hydrogen, and 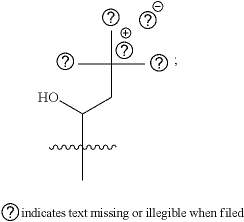
R₅, R₆, and R₇ are independently selected from the group consisting of C₁₋₁₂ alkyl, C₅₋₁₀ aryl, and 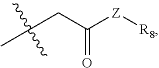
wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl, wherein alkyl and aryl are optionally substituted with halogen, C₁₋₁₂ alkyl, and C₅₋₁₀ aryl, and Z is O or NH;
R₃ is selected from the group consisting of hydrogen, and —COR₉;
R₉ is selected from the group consisting of C₁₋₁₆ alkyl, and C₅₋₁₀ aryl, wherein alkyl and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein,
degree of substitution of X in the compound of Formula I is in the range of 20-100%;
degree of substitution of each R₂ and R₄ with hydrogen, or 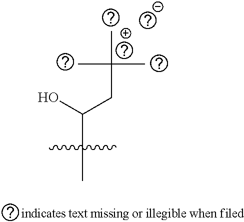
in the compound of Formula I is in the range of 20-100%;
degree of substitution of R₃ with hydrogen or —COR₉ in the compound of Formula I is in the range of 20-100%.

2. A polymer network comprising a compound of Formula I 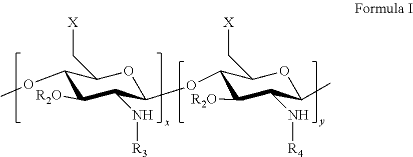
cross-linked to a compound selected from the group consisting of a compound of Formula II; 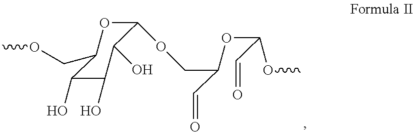
hyaluronate aldehyde, alginate aldehyde, dextran aldehyde, starch aldehyde, and chitosan aldehyde,
wherein,
X is OR₁;
R₁ is selected from the group consisting of hydrogen, and 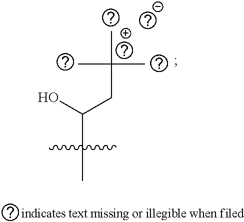
R₂ and R₄ are independently selected from the group consisting of hydrogen, and 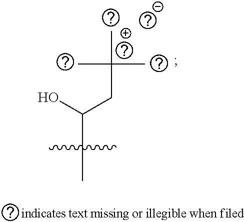
R₅, R₆ and R₇ are independently selected from the group consisting of C₁₋₁₂ alkyl, C₅₋₁₀ aryl, and 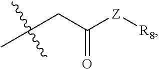
wherein alkyl and aryl are optionally substituted with halogen, C₁₋₁₂ alkyl, and C₅₋₁₀ aryl;
R₈ is selected from the group consisting of C₁₋₁₂ alkyl and C₅₋₁₀ aryl, wherein alkyl and aryl are optionally substituted with halogen, C₁₋₁₂ alkyl, and C₅₋₁₀ aryl, and Z is O or NH;
R₃ is selected from the group consisting of hydrogen and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein,
degree of substitution of X in the compound of Formula I is in the range of 20-100%;
degree of substitution of each R₂ and R₄ with hydrogen, or 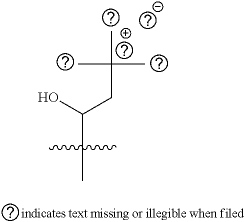
in the compound of Formula I is in the range of 20-100%;
degree of substitution of R₃ with hydrogen or —COR₉ in the compound of Formula I is in the range of 20-100%.

3. A polymer network comprising a compound of Formula I 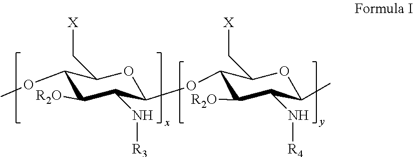
cross-linked to a compound selected from the group consisting of a compound of Formula II; 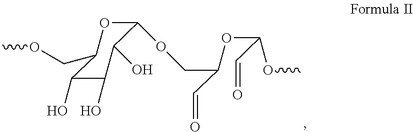
hyaluronate aldehyde, alginate aldehyde, dextran aldehyde, starch aldehyde, and chitosan aldehyde
wherein;
X is 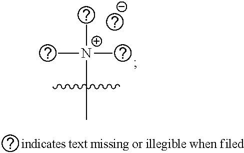
R₂ and R₄ are independently selected from the group consisting of hydrogen, and 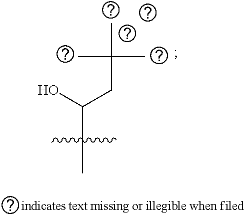 [SMILES: *C(O)CC(C)(C)C]
R₅, R₆ and R₇ are independently selected from the group consisting of C₁₋₁₂ alkyl, C₅₋₁₀ aryl, and 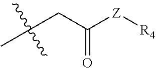
wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is selected from the group consisting of hydrogen, and —COR₉;
R₉ is selected from the group consisting of C₁₋₁₆ alkyl, and C₅₋₁₀ aryl, wherein alkyl and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of each R₂ and R₄ with hydrogen, or 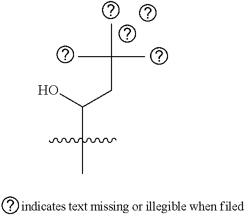 [SMILES: *C(O)CC(C)(C)C]
in the compound of Formula I is in the range of 20-100%;
degree of substitution of R₃ with hydrogen or —COR₉ in the compound of Formula I is in the range of 20-100%.

4. A polymer network comprising a compound of Formula I 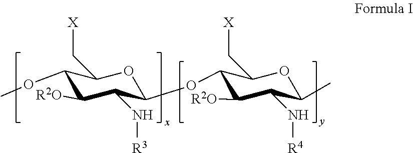
cross-linked to a compound selected from the group consisting of a compound of Formula II; 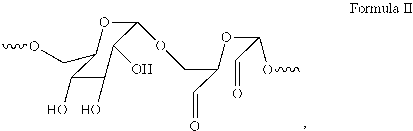
hyaluronate aldehyde, alginate aldehyde, dextran aldehyde, starch aldehyde, and chitosan aldehyde,
wherein;
X is OR₁;
R₁ is selected from the group consisting of hydrogen, and 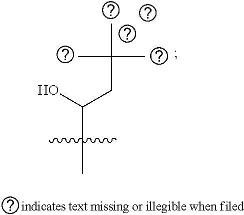 [SMILES: *C(O)CC(C)(C)C]
R₂ is selected from the group consisting of hydrogen, and 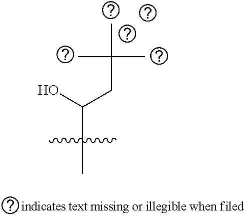 [SMILES: *C(O)CC(C)(C)C]
R₄ is 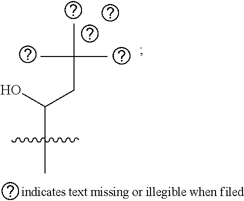 [SMILES: *C(O)CC(C)(C)C]
R₅, R₆ and R₇ are independently selected from the group consisting of C₁₋₁₂ alkyl, C₅₋₁₀ aryl, and 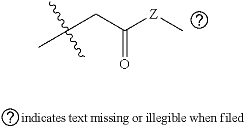
wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is selected from the group consisting of hydrogen, and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of R₂ with hydrogen or 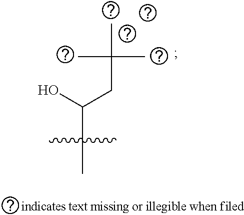 [SMILES: *C(O)CC(C)(C)C]
in the compound of Formula I is in the range of 20-100%;
degree of substitution of R₄ with 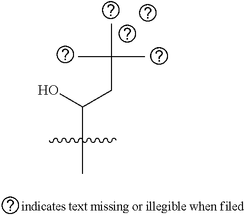 [SMILES: *C(O)CC(C)(C)C]
in the compound of Formula I is in the range of 20-100%;
degree of substitution of R₃ with hydrogen or —COR₉ in the compound of Formula I is in the range of 20-100%.

5. A polymer network comprising a compound of Formula I 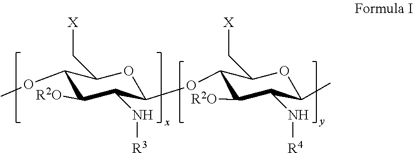
cross-linked to a compound selected from the group consisting of a compound of Formula II; 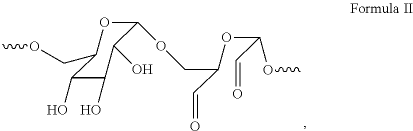
hyaluronate aldehyde, alginate aldehyde, dextran aldehyde, starch aldehyde, and chitosan aldehyde,
wherein;
X is OR₁;
R₁ is selected from the group consisting of hydrogen, and 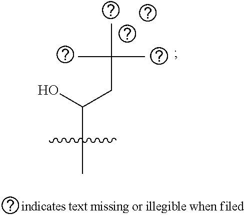 [SMILES: *C(O)CC(C)(C)C]
R₂ is hydrogen;
R₄ is selected from the group consisting of hydrogen, and 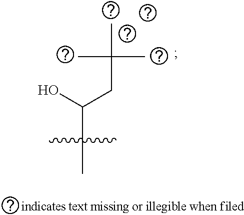 [SMILES: *C(O)CC(C)(C)C]
R₅, R₆ and R₇ are independently selected from the group consisting of C₁₋₁₂ alkyl, C₅₋₁₀ aryl, and 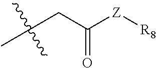
wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl and aryl are optionally substituted with halogen, C₁₋₁₂ alkyl, and C₅₋₁₀ aryl and Z is O or NH;
R₃ is selected from the group consisting of hydrogen, and —COR₉;
R₉ is selected from C₁₋₁₆ alkyl, and C₅₋₁₀ aryl, wherein alkyl and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of R₃ with hydrogen or —COR₉ in the compound of Formula I is in the range of 20-100%;
degree of substitution of R₄ with hydrogen or 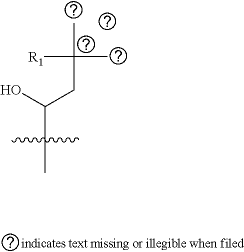
in the compound of Formula I is in the range of 20-100%.

6. A polymer network comprising a compound of Formula I 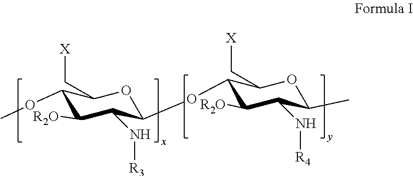
cross-linked to a compound selected from the group consisting of a compound of Formula II; 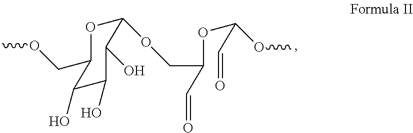
hyaluronate aldehyde, alginate aldehyde, dextran aldehyde, starch aldehyde, and chitosan aldehyde,
wherein;
X is OR₁;
R₁ is hydrogen;
R₂ is hydrogen;
R₄ is 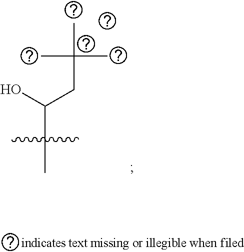 [SMILES: *C(O)CC(C)(C)C]
R₅, R₆ and R₇ are independently selected from the group consisting of C₁₋₁₂ alkyl, C₅₋₁₀ aryl, and 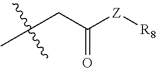
wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is hydrogen;
A^{⊖} is negatively charged counter anion;
x is 1 to 1000;
y is 1 to 1000;
wherein;
degree of substitution of R₄ with 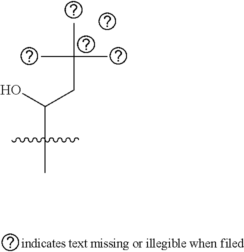 [SMILES: *C(O)CC(C)(C)C]
in the compound of Formula I is in the range of 20-100%.

7. A polymer network comprising a compound of Formula I 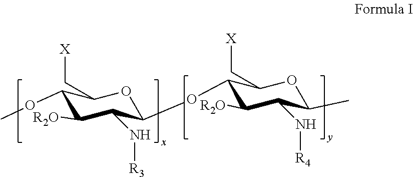
cross-linked to a compound selected from the group consisting of a compound of Formula II; 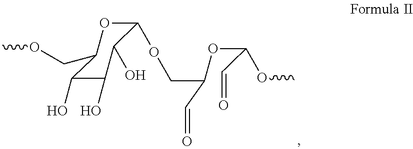
hyaluronate aldehyde, alginate aldehyde, dextran aldehyde, starch aldehyde, and chitosan aldehyde,
wherein;
X is OR₁;
R₁ is hydrogen;
R₂ is hydrogen
R₄ is 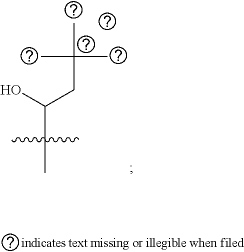 [SMILES: *C(O)CC(C)(C)C]
R₅, R₆, and R₇ are independently selected from the group consisting of C₁₋₁₂ alkyl, C₅₋₁₀ aryl, and 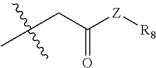
wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl and aryl are optionally substituted with halogen, C₁₋₁₂ alkyl, and C₅₋₁₀ aryl and Z is O or NH;
R₃ is-COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
P is negatively charged counter anion;
x is 1 to 1000;
y is 1 to 1000;
wherein;
degree of substitution of R₃ with —COR₉ in the compound of Formula I is in the range of 20-100%;
degree of substitution of R₄ with 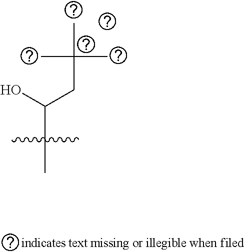 [SMILES: *C(O)CC(C)(C)C]
in the compound of Formula I is in the range of 20-100%.

8. A polymer network comprising a compound of Formula I 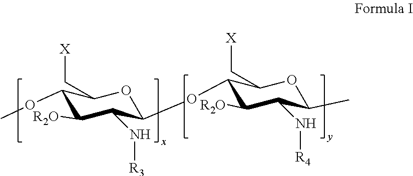
cross-linked to a compound selected from the group consisting of a compound of Formula II; 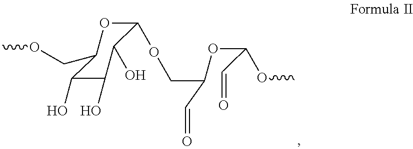
hyaluronate aldehyde, alginate aldehyde, dextran aldehyde, starch aldehyde, and chitosan aldehyde,
wherein;
X is 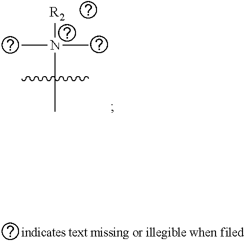
R₂ is hydrogen;
R₄ is selected from the group consisting of hydrogen, and 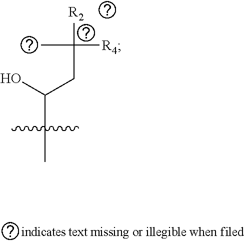
R₅, R₆ and R₇ are independently selected from the group consisting of C₁₋₁₂ alkyl, C₅₋₁₀ aryl, and 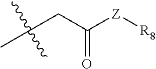
wherein alkyl and aryl are optionally substituted with halogen, C₁₋₁₂ alkyl, and C₅₋₁₀ aryl;
R₈ is selected from C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is selected from the group consisting of hydrogen and —COR₉;
R₉ is selected from the group consisting of C₁₋₁₂ alkyl, and C₆₋₁₀ aryl, wherein alkyl, and aryl are optionally substituted with halogen, C₁₋₁₂ alkyl, and C₆₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of R₃ with hydrogen or —COR₉ in the compound of Formula I is in the range of 20-100%;
degree of substitution of R₄ with hydrogen or 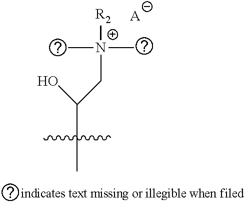
in the compound of Formula I is in the range of 20-100%.

9. A polymer network comprising a compound of Formula I 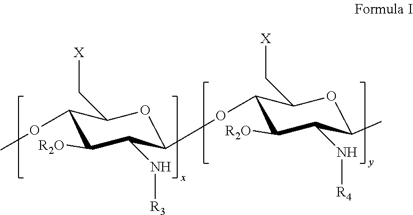
cross-linked to a compound selected from the group consisting of a compound of Formula II; 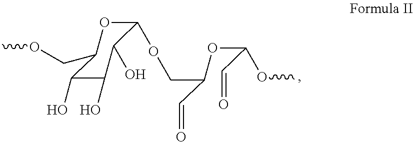
hyaluronate aldehyde, alginate aldehyde, dextran aldehyde, starch aldehyde, and chitosan aldehyde,
wherein;
X is 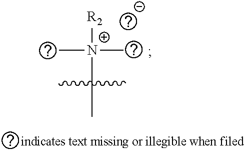
R₂ is selected from the group consisting of hydrogen and 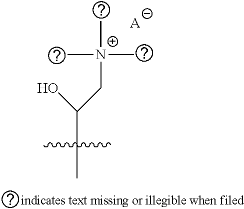
R₄ is hydrogen;
R₅, R₆ and R₇ are independently selected from the group consisting of C₁₋₁₂ alkyl, C₅₋₁₀ aryl, and 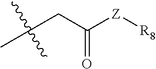
wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is hydrogen;
P is negatively charged counter anion;
x is 1 to 1000;
y is 1 to 1000;
wherein;
degree of substitution of X in the compound of Formula I is in the range of 20-100%;
degree of substitution of R₂ with hydrogen or 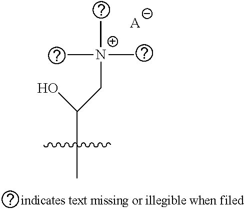
in the compound of Formula I is in the range of 20-100%.

10. A polymer network comprising a compound of Formula I 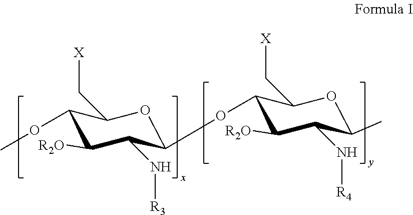
cross-linked to a compound selected from the group consisting of a compound of Formula II; 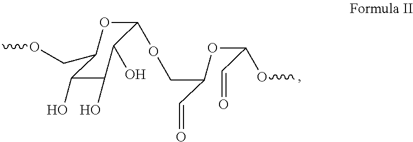
hyaluronate aldehyde, alginate aldehyde, dextran aldehyde, starch aldehyde, and chitosan aldehyde,
wherein;
X is 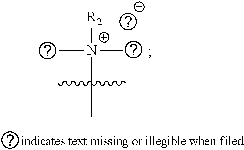
R₂ is hydrogen;
R is 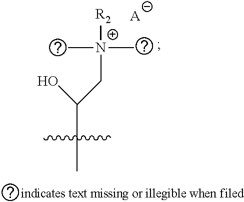
R₅, R₆ and R₇ are independently selected from the group consisting of C₁₋₁₂ alkyl, C₅₋₁₀ aryl, and 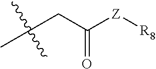
wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is hydrogen;
A^{⊖} is negatively charged counter anion;
x is 1 to 1000;
y is 1 to 1000;
wherein;
degree of substitution of X in the compound of Formula I is in the range of 20-100%;
degree of substitution of R₄ with 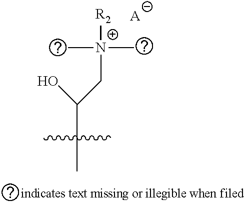
in the compound of Formula I is in the range of 20-100%.

11. A polymer network comprising a compound of Formula I 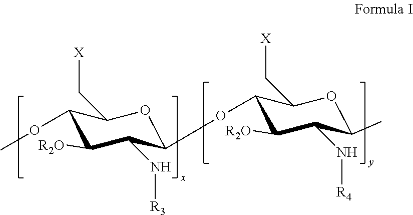
cross-linked to a compound selected from the group consisting of a compound of Formula II; 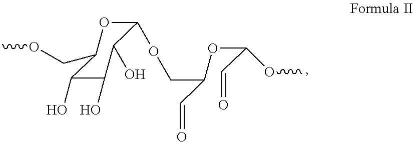
hyaluronate aldehyde, alginate aldehyde, dextran aldehyde, starch aldehyde, and chitosan aldehyde,
wherein;
X is 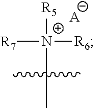
R₂ is hydrogen;
R₄ is 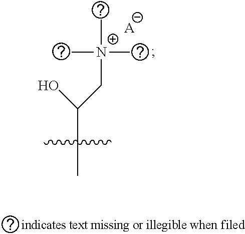
R₅, R₆ and R₇ are independently selected from the group consisting of C₁₋₁₂ alkyl, C₅₋₁₀ aryl, and 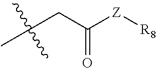
wherein alkyl, and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl and Z is O or NH;
R₃ is —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
wherein;
degree of substitution of X in the compound of Formula I is in the range of 20-100%;
degree of substitution of R₃ with —COR₉ in the compound of Formula I is in the range of 20-100%;
the degree of substitution of R₄ with 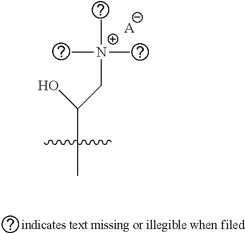
in the compound of Formula I is in the range of 20-100%.

12. A polymer network comprising a compound of Formula I 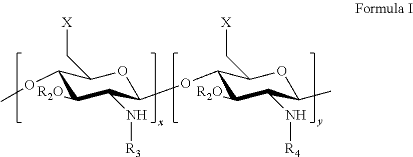
cross-linked to a compound selected from the group consisting of a compound of Formula II; 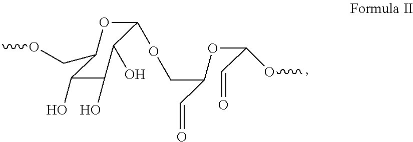
hyaluronate aldehyde, alginate aldehyde, dextran aldehyde, starch aldehyde, and chitosan aldehyde,
wherein;
X is OR₁;
R₁ is hydrogen;
R₂ is hydrogen;
R₄ is 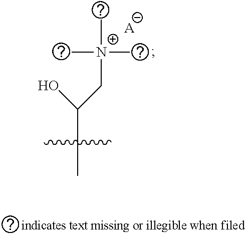
R₅, R₆, and R₇ are independently substituted with C₁₋₁₂ alkyl;
R₃ is hydrogen;
A^{⊖} is negatively charged counter anion;
x is 1 to 1000;
y is 1 to 1000;
wherein;
degree of substitution of R₄ with 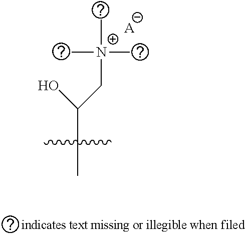
in the compound of Formula I is in the range of 20-100%.

13. The polymer network as claimed in claims 1 to 12, wherein A^{⊖} is selected from the group consisting of Cl⁻, Br⁻, I⁻, OH⁻, HCO³⁻, CO₃²⁻, R₁₀COO⁻, R₁₀SO₄⁻, and R₁₀SO₃⁻, wherein R₈ is selected from the group consisting of hydrogen, C₁₋₆ alkyl, and C₅₋₁₀ aryl, wherein C₁₋₆ alkyl, and C₅₋₁₀ aryl are optionally substituted with hydroxyl, nitro, halogen, alkyl, aryl, —COOR₈.

14. The polymer network as claimed in claims 1 to 12, wherein the compound of Formula II is cross linked to the compound of Formula I through aldehyde group of Formula II and the amine group of Formula I.

15. The polymer network as claimed in claim 1, wherein the compound of Formula I is N-(2-hydroxy)-propyl-3-trimethylammonium chitosan chloride.

16. A process of the preparation of a polymer network as claimed in claim 1.

17. The polymer network as claimed in any of the claims 1-12 for use in antimicrobial infections.

18. The polymer network as claimed in claim 17 for use as antimicrobial agents in the treatment of diseases caused by bacteria, fungi, and virus.

19. The polymer network as claimed in any of the claims 1-12 for use as antibacterial agents in the treatment of diseases caused by Gram-positive, Gram-negative bacteria or drug-resistant bacteria.

20. A composition comprising the polymer network as claimed in any of the claims 1 to 12 in an aqueous solution.

21. A composition comprising the polymer network as claimed in claim 1 to 12 in an aqueous solution and buffer solution.

22. The composition as claimed in claim 20 or 21, wherein the compound of Formula I is in the range of 0.5% to 15% w/w of the total composition and the compound of Formula II is in the range of 2% to 10% w/w of the total composition.

23. The composition as claimed in claim 20 or 21, wherein the compound of Formula I is in the range of 0.5% to 2.5% w/w of the total composition and the compound of Formula II is in the range of 2% to 3% w/w of the total composition.

24. The composition as claimed in claim 20 or 21, wherein the compound of Formula I is in the range of 1% to 2.5% w/w of the total composition and the compound of Formula II is 2.5% w/w of the total composition.

25. The composition as claimed in claim 20 or 21, wherein the compound of Formula I is 2.5% w/w of the total composition and the compound of Formula II is 2.5% w/w of the total composition.

26. The composition as claimed in any of the claims 20 to 25, wherein the compound of Formula II is N-(2-hydroxy)-propyl-3-trimethylammonium chitosan chloride.

27. A hydrogel comprising a polymer matrix as claimed in claim 1, and water.

28. The hydrogel as claimed in claim 27, wherein the compound of Formula I is in the range of 2% to 15% w/w of the total composition and the compound of Formula II is in the range of 0.5% to 10% w/w of the total composition.

29. The hydrogel as claimed in claim 27, wherein the compound of Formula I is in the range of 2% to 3% w/w of the total composition and the compound of Formula II is in the range of 0.5% to 2.5% w/w of the total composition.

30. The hydrogel as claimed in claim 27, wherein the compound of Formula I is in the range of 1% to 2.5% w/w of the total composition and the compound of Formula II is 2.5% w/w of the total composition.

31. The hydrogel as claimed in claim 27, wherein the compound of Formula I is 2.5% w/w of the total composition and the compound of Formula II is 2.5% w/w of the total composition.

32. The hydrogel as claimed in claim 27 to 31, wherein the compound of Formula I is N-(2-hydroxy)-propyl-3-trimethylammonium chitosan chloride.

33. The hydrogel as claimed in claim 27 comprises one or more biologically active agents.

34. The hydrogel as claimed in claim 33, wherein the biologically active agent is selected from antibiotics, silver nanoparticle, analgesic, anti-inflammatory drugs and growth factor such as human recombinant bone morphogenetic protein.

35. A process of preparing a hydrogel as claimed in claim 27, the process comprising:
contacting a compound of Formula I, 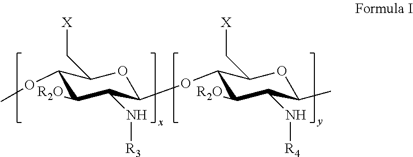
with the compound of Formula II; 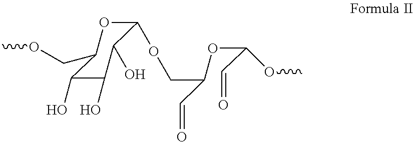
wherein;
X is selected from the group consisting of OR₁, and 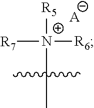
R₁ is selected from the group consisting of hydrogen, and 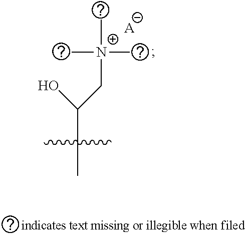
R₂ and R₄ are independently selected from the group consisting of hydrogen, and 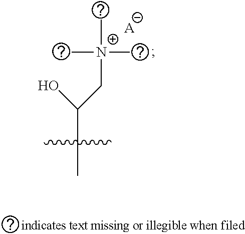
R₅, R₆ and R₇ are independently selected from the group consisting of C₁₋₁₂ alkyl, C₅₋₁₀ aryl, and 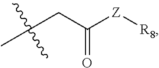
wherein alkyl and aryl are optionally substituted with halogen, C₁₋₁₂ alkyl, and C₅₋₁₀ aryl;
R₈ is selected from the group consisting of C₁₋₁₂ alkyl, and C₅₋₁₀ aryl wherein alkyl, and aryl are optionally substituted with halogen, C₁₋₁₂ alkyl, and C₅₋₁₀ aryl, and Z is O or NH;
R₃ is selected from the group consisting of hydrogen, and —COR₉;
R₉ is selected from the group consisting of C₁₋₁₆ alkyl, and C₅₋₁₀ aryl, wherein alkyl, and aryl are optionally substituted with halogen, C₁₋₁₂ alkyl, and C₅₋₁₀ aryl;
A^{⊖} is negatively charged counter anion;
x is 1 to 1000;
y is 1 to 1000;
and water optionally in presence of a buffer to obtain the hydrogels;
wherein the Formula II and Formula I are present in an amount such that the ratio of RNH₂/RCHO group is between 0.5 to 1.5.

36. The process as claimed in claim 35, wherein the buffer is phosphate buffer.

37. A method of treating a condition mediated by one or more microbial agents, comprising administering to a subject suffering from a condition mediated by one or more microbial agents a therapeutically effective amount of the hydrogel according to any one of claims 27 to 34 or the composition according to claim 20 or claim 21.

38. A method for repairing soft tissue, said method comprising the step of administering the hydrogel according to any one of claims 27 to 34 or the composition according to claim 20 or claim 21 at the site of a soft tissue in need of repair of a patient.

39. A method for repairing or resurfacing a damaged cartilage, said method comprising the step of administering the hydrogel according to any one of claims 27 to 34 or the composition according to claim 20 or claim 21 in or around a cartilage in need of repair or resurfacing of a patient

40. Use of the hydrogel as claimed in any one of claims 27 to 34 or the composition as claimed in claim 20 or claim 21 for soft tissue repair.

41. Use of the hydrogel as claimed in any one of claims 27 to 34 or the composition as claimed in claim 20 or claim 21 for bone repair.

42. Use of the hydrogel as claimed in any one of claims 27 to 34 or the composition as claimed in claim 20 or claim 21 for repairing or resurfacing damaged cartilage.

43. Use of the hydrogel as claimed in any one of claims 27 to 34 for the manufacture of a medicament for soft tissue repair.

44. Use of the hydrogel as claimed in any one of claims 27 to 34 for the manufacture of a medicament for bone repair.

45. Use of the hydrogel as claimed in any one of claims 27 to 34 for the manufacture of a medicament for repairing or resurfacing damaged cartilage.

46. Use of the hydrogel as claimed in any one of claims 27 to 34 for the manufacture of a medicament for repairing meniscus.

47. A kit comprising;
(a) a compound of Formula I and
(b) a compound of Formula II
wherein the compound of Formula I is contacted with the compound of Formula II to obtain the polymer as claimed in claim 1

48. A kit as claimed in claim 47, wherein either or both of (a) and (b) are provided in separate aqueous solutions optionally with a buffer.

49. A kit as claimed in claim 47, wherein the aqueous solution of (a) is between 0.5% to 10% w/w and the aqueous solution of (b) is between 2% to 10% w/w.

50. A kit as claimed in claim 47, further comprises an aqueous solution to allow cross linking of (a) and (b) to occur.

51. A kit as claimed in claim 47, wherein the compound of Formula I is N-(2-hydroxy)-propyl-3-trimethylammonium chitosan chloride.

52. An antimicrobial hydrogel comprising a polymer network consisting of (2-hydroxy)-propyl-3-trimethylammonium chitosan chloride (HTCC), and a second polymer polydextran aldehyde (PDA), wherein said polymer blend solidifies to form a solid hydrogel at physiological body temperature, wherein said polymer blend is formed by a (2-hydroxy)-propyl-3-trimethylammonium chitosan chloride (HTCC) or a compound of Formula I which is cross linked to a compound selected from the group consisting of hyaluronate aldehyde, alginate aldehyde, dextran aldehyde, starch aldehyde, chitosan aldehyde and a compound of Formula II.

53. An antimicrobial hydrogel with biologically active molecules comprising a polymer network consisting of (2-hydroxy)-propyl-3-trimethylammonium chitosan chloride (HTCC) or a compound of Formula I, and a second polymer polydextran aldehyde (PDA) or a compound of Formula II along with the biologically active molecules wherein said polymer blend solidifies to form a solid hydrogel at physiological body temperature.

54. An antimicrobial hydrogel with silver nanoparticle comprising a polymer network consisting of (2-hydroxy)-propyl-3-trimethylammonium chitosan chloride (HTCC) or a compound of Formula I, and a second polymer polydextran aldehyde (PDA) or a compound of Formula II along with the preformed silver nanoparticle wherein said polymer blend solidifies to form a solid hydrogel at physiological body temperature.
